(19) United States
(12) Patent Application Publication
(10) Pub. No.: US 2022/0001077 A1
(43) Pub. Date: Jan. 6, 2022

(54) Liquid Crystalline Collagen Materials and Use in Connective Tissue Repair

(71) Applicant: Northeastern University, Boston, MA (US)

(72) Inventors: Jeffrey William RUBERTI, Lexington, MA (US); Patrick Anthony BRADLEY, Boston, MA (US)

(21) Appl. No.: 17/293,561

(22) PCT Filed: Nov. 13, 2019
(86) PCT No.: PCT/US2019/061329

§ 371 Date: May 13, 2021

Related U.S. Application Data
(60) Provisional application No. 62/760,903, filed on Nov. 13, 2018.
(60) Provisional application No. 62/800,945, filed on Feb. 4, 2019.

(51) Int. Cl. A61L 27/24; A61K 38/18; A61K 38/39; A61K 38/48; A61K 38/20; A61P 19/04; C08L 89/06; C07K 14/78; A61L 27/38; A61L 27/54; A61L 27/22; A61H 23/02

(52) U.S. Cl. CPC A61L 27/24; A61H 2201/10; A61K 38/39; A61K 38/4886; A61K 38/2006; A61P 19/04; C08L 89/06; C07K 14/78; A61L 27/3834; A61L 27/3843; A61L 27/386; A61L 27/54; A61L 27/3895; A61L 27/227; A61H 23/0245; A61L 2300/236; A61L 2300/252; A61L 2300/41; A61L 2300/412; A61L 2400/12; A61L 2430/10; A61K 38/1841

(57) ABSTRACT

Compositions and methods are provided to accelerate and improve wound repair and reconstruction of connective tissue structures, including tendons, by assembly of collagen using liquid crystalline collagen. The compositions and methods can be used to treat various forms of connective tissue injury or to prevent or slow degeneration to vulnerable tendons that are generally refractory to repair.

### STATEMENT REGARDING FEDERALLY SPONSORED RESEARCH OR DEVELOPMENT

This invention was made with government support under Grant No. 1309579 awarded by the National Science Foundation. The government has certain rights in the invention.

### BACKGROUND

More than 500,000 surgical procedures are performed each year for damaged ligaments or tendons, and these connective tissue (CT) surgeries have long recovery times. CT injuries are of great concern to individuals with physically demanding professions, active lifestyles, or the elderly. CT injuries heal slowly and often incompletely, resulting in considerable pain, suffering and a decrease in quality of life. Quadriceps, Achilles, rotator cuff, and biceps tendons are the most common tendon injuries, with ruptures of the supraspinatus tendon in the rotator cuff affecting an estimated 22.1% of the population. Tendon rupture can occur from acute injury or through chronic degradation of the tissue in the absence of an apparent injury via an unknown biological mechanism. This lack of knowledge that underlies tendon breakdown results in limited and inadequate management options for tendon disorders. Clinical guidelines for a ruptured Achilles tendon recommend 6-12 weeks of immobilization and up to 4 months of recovery, while rotator cuff repairs are generally recalcitrant. Despite intense scientific focus, there has been little improvement in the rate or quality of tendon or ligament repair. A major advance in the repair of damaged CT would be a substantial reduction in healing time and an increase in the strength of repair.

In the United States alone, there are over 100 million injury-related medical appointments each year, with 60-67% specific to musculoskeletal injury. The direct healthcare expense for musculoskeletal conditions is approximately $130 billion annually. Many of the soft tissue musculoskeletal injuries involve tendons and ligaments and often require surgical intervention. Perhaps the most staggering statistic is that over 288 million workdays are lost annually due to musculoskeletal conditions, accounting for 73% of total work days lost. Compensation, lost wages, and lost productivity further burden the economy by approximately $50 billion every year. Workdays lost to slow or incomplete healing are a major issue. For example, the most common medical recommendation following tendon rupture is four months of recovery for those performing manual labor. Studies have shown that the mechanical properties of injured CT may actually take years to approach the properties of the uninjured state. The prevalence of rotator cuff injuries is another source of concern. Rotator cuff tears affect 40% or more of patients older than 60 years and are common causes of debilitating pain, reduced shoulder function, and muscle weakness. Repeat surgeries that are intended to address re-tears or re-ruptures are another area with significant potential for improvement. When repaired, rotator cuffs often form a recurrent tendon re-tear after surgery, often attributed to the patient's age or health, the size of the tear, or the level of tendon degeneration prior to surgery. Given the significant numbers of people negatively affected by tendon damage and the clear deficiencies in outcome, particularly with rotator cuff damage, new approaches should be explored.

Collagen is the predominant extracellular matrix molecule in load-bearing tissues. An important characteristic of collagen type I is its well-understood hierarchical structure from the nanoscale to the macroscale. Organizational motifs of collagen are very tissue specific, with the morphology reflecting the mechanical environment. The intricate and highly organized architecture of collagen materials leads to specific physical and biological functions that is characteristic across length scales. The self-assembly properties and biocompatibility of the collagen molecule make it a premier candidate for tissue engineering and regenerative medicine.

Current therapeutic approaches to damaged or diseased tendon involve implantation of rehydrated type I collagen gels, occasionally alongside the delivery of embedded stem cells or therapeutics. This collagenous network is sutured onto the damaged tendon where host cells infiltrate and remodel the implant. Since the implant is sutured directly onto the tendon, it also functions to increase the effective diameter of the tendon and therefore decrease the peak strain of the host tissue. This is hypothesized to protect the tendon from further tear progression as it undergoes remodeling. However, despite advances in fabrication and implantation techniques of these scaffolds, complete healing of collagenous tissues remains elusive, with only marginal increases in clinical scores and significant recurrence of wounds and tears. This lack of healing has been attributed to the paucity of native cell populations and their inability to mount a repair response. Reliance on scarce host cells to infiltrate and remodel these implants, even in the presence of numerous growth factors, is therefore insufficient.

### SUMMARY

The present technology provides compositions and methods for the controlled delivery of exogenous collagen to damaged connective tissue. The compositions and methods can be used to accelerate and improve wound repair by self-assembly of collagen. The compositions are provided in form of both stable and metastable liquid crystal collagen, which can form various networks or scaffolds. The stable and metastable liquid crystal collagen compositions can be formulated into injectable particles or gels. Injections of these materials can be used to treat various forms of connective tissue injury, or to prevent or slow degeneration secondary to cellular senescence for particularly vulnerable tendons that are generally refractory to repair when they fail (e.g. supraspinatus, flexor tendons, and Achilles tendon).

The present technology utilizes new materials to accelerate connective tissue repair by the controlled delivery of active collagen (i.e., tropocollagen monomer). Active collagen-mediated repair utilizes data and a theory, which show that collagen fibrillogenesis and stability are promoted by mechanical force. The concept that force directly drives collagen fiber formation (referred to as Mechanochemical Force Structure Causality or MFSC) suggests that if there is sufficient available active collagen (tropocollagen), and intermittent tensile mechanical forces are applied to damaged tissue, then repair fibrils are formed, damaged fibrils are repaired, and both are retained preferentially in the path of the load. The premise of MFSC has been clearly demonstrated in a cell-free system.

Collagen retention and assembly occur in the path of mechanical force. Retention is the effect whereby strain stabilizes collagen against thermal and enzymatic degradation. Mechanical strain can directly drive collagen assembly along force lines. Together, these two effects make it possible to provide sufficient active collagen to an injury or defect undergoing mechanical perturbation (e.g. ambulation, physical therapy, controlled passive loading, electromyography, ultrasound, vibration therapy, etc.) which then leads to faster injury repair and increased organization of the repaired tissue. It is important, however, to provide sufficient active collagen to the defect edges to facilitate recruitment and polymerization via flow-induced crystallization (FIC) or preferential incorporation of collagen molecules into damaged tissue. FIGS. 1A-1D show how extensional force applied to a 15 mg/ml solution of type I collagen molecules causes highly-aligned collagen fiber formation and facilitates the repair of a broken fiber.

An aspect of the present technology is the development of a highly-densified, liquid crystal collagen material with high stability (stable liquid crystal collagen, or SLCC), or a scaffold containing such material (stable liquid crystal collagen scaffold, or SLCCS). The inventors have generated collagenous sheets with controlled fibril alignment that persist over 7 orders of magnitude in length scale and which possess collagen densities that are about 3-4 times that of native tendon (>800 mg/ml). FIGS. 2A-2E show multiple levels of organization and the persistence of that organization pattern over long distances (well over a centimeter in this case). The material is robust (suturable) even when very thin, will maintain collagen in its active form (no cross-links), and keep it retained within the stable structure for long periods.

Another aspect of the technology is the development of a highly-densified, metastable liquid crystal collagen (MLCC) material with tunable stability, or a scaffold containing such material (MLCCS). The material shown in FIGS. 2A-2E can be tuned to release active collagen at different rates by modulating the process that packs the monomers into the stable structure. The difference between the processing of SLCC and MLCC is the duration of the application of ultrasonic and osmotic forces. The structure of MLCC is less stable and also less dense than the stable collagen sheet, ˜2 times the collagen content of tendon. While this material has similar long range and local organization as the more stable collagen, the fibrils do not possess D-periodicity, suggesting they are not in their lowest natural energy state (FIG. 3A). Quantitatively, the unstable collagen shows a burst release profile in PBS (FIG. 3B).

Both SLCC and MLCC can be introduced into a damaged connective tissue of a mammal, including a human, where they can act as organizational guidance structures to direct the repaid of aligned tissues, such as collagen-containing connective tissue. In particular, they can be used to aid in tendon repair by providing or guiding replacement of all or part of missing or damaged tissue.

Still another aspect of the invention is a bi-stable active collagen patch. A strong, bi-stable patch can be formed by fusing sheets of SLCC & MLCC collagen (FIG. 4). The sheet can be sutured to the supraspinatus tendon, for example. Because of its strength and density, the outer, stable sheet (SLCC) contains the active collagen released from the inner, less stable sheet (MLCC). Using this approach active collagen can be retained adjacent to the treatment site where it can accelerate the repair of the defect by raising the collagen concentration and provide a second, slower release rate of collagen into the wound for longer term repair. Patches comprising more than two layers of collagen MLCCs and SLCCs can be generated to produce systems with highly-controlled release of collagen over long periods.

Yet another aspect of the technology is a process of creating SLCCS by a process termed “osmotic breathing”. For example, collagen can be dehydrated for 12 hours at 4° C. against acidic 10% polyethylene glycol dissolved in deionized water. The collagen solution can then be placed into a fresh dialysis bag which is placed in acidic 40% polyethylene glycol dissolved in deionized water. The collagen solutions can be subjected to ultrasound for 30 minutes, twice per day, over the course of 7 days. The dialysis bags can then be transferred to 40% polyethylene glycol dissolved in 1×PBS at 7.4 pH at 35° C. and then to 1×PBS solution at 35° C. at 24-hour intervals over the course of 7 days.

Another aspect of the technology is a process of creating MLCCS by osmotic breathing. The process can include first dehydrating a collagen solution by dialysis against acidic 10% polyethylene glycol dissolved in deionized water for 12 hours at 4° C. The solution is then placed into a fresh dialysis bag which is placed into acidic 40% polyethylene glycol dissolved in deionized water for 7 days at 4° C. The dialysis bag is then transferred to neutralized 40% polyethylene glycol dissolved in 1×PBS for 4 days at 4° C. The PEG/collagen solution is then warmed to 37° C. to initiate fibrillogenesis for 48 hours.

Still another aspect of the technology is a process of fabricating MLCCS via molecular crowding, in which liquid crystal phasing of collagen mesogens is developed through dialysis against, for example, acidic 40% polyethylene glycol (PEG). Once the mesogen concentration stabilizes (at about 300 mg/mL), “metastable fibrillogenesis” is induced via exposure to PEG/1×PBS at pH=7.4. In some embodiments, SLCCS can be produced by a similar process but with two procedural changes: 1) During the initial dialysis, the densifying collagen solution is subjected to two 30-minute treatments per day of, for example, 130 W ultrasound and 2) After 7 days, the sample is subjected to a 7-day PEG-PBS cyclic “osmotic breathing” treatment comprising alternating submersion into neutralized concentrated PEG-1×PBS and only 1×PBS at 37° C. at 24-hour intervals to induce “stable fibrillogenesis”.

Yet another aspect of the technology is a process of assembling a combined MLCC/SLCC bi-stable patch by polymerizing a dense (e.g., 15 mg/mL) solution of neutralized collagen between the MLCC and SLCC sheets and subjecting the patch to neutral 40% PEG-1×PBS at 37° C. for 24-hours. In some embodiments, UVA cross-linking can then be utilized, and SLCCs are cross-linked by exposure to UV and/or riboflavin per a modified Dresden protocol. An original cross-linking procedure, commonly referred to as the Dresden protocol, calls for UVA exposure for 30 minutes at 3 mW/cm⁻². UV exposure time from 10 to 40 minutes is used to achieve a range of cross-linking densities as assessed by denaturation temperature. In some embodiments, for each class of material produced, sheets are cut such that long-axis is perpendicular to the birefringent texture (parallel to fibril orientation).

In a further process of the technology, the release rates of MLCC are tuned by altering the duration of ultrasound (US) and/or other conditions. In some embodiments, MLCC and SLCC are implanted or injected (after being fragmented) to serve as a tissue filler in a subject. In some embodiments, ultrasound is applied to the subject to convert the MLCC to SLCC and fuse the particles into a more solid, degradation-resistant state that possesses an overall lower energy.

The present invention is further summarized by the following list of items:

1. A non-naturally occurring, stable, liquid crystal collagen composition, wherein the composition comprises type I, II, III, V, and/or XI collagen organized into fibril-like structures 50-200 nm in diameter, wherein the fibril-like structures are locally aligned along their longitudinal axis and have D-periodic banding structure larger than about 67 nm, and wherein the composition has a density of at least about 500 mg/mL.
2. The collagen composition of 1 above, wherein the fibril-like structures shed monomeric collagen at a rate less than 1% per day at 37° C.
3. The collagen composition of 1 above, wherein the composition has a density of about 800 mg/m L.
4. A non-naturally occurring, metastable, liquid crystal collagen composition, wherein the composition comprises type I, II, III, V, and/or XI collagen organized into fibril-like structures 50-100 nm in diameter, wherein the fibrils are locally aligned along their longitudinal axis and substantially lack native D-periodic banding structure, and wherein the composition has a density of about 200-400 mg/mL.
5. A bi-stable liquid crystal collagen composition comprising the composition of 1 above and the composition of 4 above.
6. The bi-stable liquid crystal collagen composition of 5 above, wherein the relative amounts of the composition of 1 and the composition of 4 are selected to provide a desired collagen delivery rate in a collagen assembly process.
7. The composition of any one of 1-6 above configured as a plurality of particles, a gel, or a tissue scaffold.
8. The composition of 7 above configured as a tissue scaffold, wherein the scaffold further comprises one or more types of cells and/or one or more therapeutic agents.
9. The composition of 8 above, wherein the one or more therapeutic agents are selected from the group consisting of anti-inflammatory molecules, matrix anabolic molecules (signaling molecules which drive matrix assembly e.g. TGF-β3 and molecules directly involved in matrix construction e.g. fibronectin), matrix catabolic molecules (signaling molecules which drive matrix destruction e.g. IL-1 and molecules directly involved in matrix destruction e.g. matrix metalloproteases), proteoglycans, and glycosaminoglycans.
10. A method of making a stable liquid crystalline collagen composition, comprising the steps of: (a) concentrating a solution comprising type I, II, III, V, and/or XI collagen under acidic conditions; (b) subjecting the product of (a) to periodic sonication for several days; (c) osmotically cycling, neutralizing, and warming the product of (b) to form the stable liquid crystalline collagen composition.
11. The method of 10 above, wherein step (a) comprises dialyzing the collagen solution against an aqueous solution comprising about 10% polyethylene glycol having a molecular weight of about 35000 Daltons at a pH of about 1.5-2.5.
12. The method of 11 above, wherein step (a) comprises concentrating the collagen solution to about 10 to about 15 mg/mL.
13. The method of 11 or 12 above, further comprising the step of: (a1) dialyzing the product of step (a) against an aqueous solution comprising about 40% polyethylene glycol having a molecular weight of about 35000 Daltons at a pH of about 1.5-2.5 for about 7 days.
14. The method of any of 10-13 above, wherein step (a) is performed at about 4° C.
15. The method of 10 above, wherein step (b) comprises subjecting the concentrating collagen solution to sonication at 50-500 watts power, such as 130 watts power, and 5 kHz-50 kHz, such as 20 kHz, for 5-120 minutes from 1-5 times/day, such as 30 minutes twice per day.
16. The method of 10 above, wherein the osmotic cycling of step (c) comprises alternating dialysis of the concentrating collagen solution against (i) 40% polyethylene glycol in phosphate buffered saline at pH 7.4 and (ii) phosphate buffered saline at pH 7.4.
17. The method of any of 10-16 above, wherein warming of step (c) comprises performing said osmotic cycling and neutralization at about 35° C.
18. The method of any of 10-17 above, wherein the stable liquid crystalline collagen is stable under conditions of mechanical load, enzymatic cleavage, and solvent dissolution.
19. A method of making a metastable liquid crystalline collagen composition, comprising the steps of: (a) concentrating a solution comprising type I, II, III, V, and/or XI collagen under acidic conditions; (b) neutralizing and warming the product of (a) to form the metastable liquid crystalline collagen composition.
20. The method of 19 above, wherein step (a) comprises dialyzing the collagen solution against an aqueous solution comprising about 10% polyethylene glycol having a molecular weight of about 35000 Daltons at a pH of about 1.5-2.5.
21. The method of 20 above, wherein step (a) comprises concentrating the collagen solution to about 10 to about 15 mg/mL.
22. The method of 20 or 21 above, further comprising the step of: (a1) dialyzing the product of step (a) against an aqueous solution comprising about 40% polyethylene glycol having a molecular weight of about 35000 Daltons at a pH of about 1.5-2.5 for about 7 days.
23. The method of any of 19-22 above, wherein step (a) is performed at about 4° C.
24. The method of any of 19-23 above, wherein step (b) comprises dialysis of the concentrating collagen solution against 40% polyethylene glycol in phosphate buffered saline at pH 7.4.
25. The method of any of 19-24 above, wherein the warming of step (b) comprises performing said neutralization at about 35° C.
26. The method of any of 10-25 above, further comprising adding one or more anti-inflammatory molecules, matrix anabolic molecules, matrix catabolic molecules, proteoglycans, and/or glycosaminoglycans to the collagen solution at any step of the method.
27. The method of any of 10-26 above, further comprising applying tensile loading to the composition at any step of the method, optionally with the addition of monomeric type I, II, III, V, and/or XI collagen to the composition.
28. A method to aid in repairing damaged connective tissue in a human or other mammalian subject, the method comprising the steps of:

(a) providing a liquid crystalline collagen composition of any of claims 1-9; and

(b) placing said composition into a region comprising damaged connective tissue in the subject;

whereby said damaged connective tissue is at least partially repaired.
29. The method of 28, wherein step (a) comprises:

(a1) obtaining cells from the subject;

(a2) culturing the cells and optionally inducing differentiation and/or proliferation of the cells and/or collagen secretion by the cells;

(a3) harvesting collagen from the culture; and

(a4) forming a liquid crystalline collagen composition of any of claims 1-9 using the harvested collagen.

30. The method of 28 or 29, further comprising the step of:

(c) performing mechanical stimulatory therapy of said region.

31. The method of 30, wherein said mechanical stimulatory therapy comprises one or more of continuous passive motion, ultrasound, vibration, and electromyostimulation.
32. The method of any of 28-31, wherein step (b) is performed continuously or intermittently over a period of time.
33. The method of any of 28-32, wherein the liquid crystalline collagen composition further comprises one or more additional extracellular matrix molecular component.
34. The method of 33, wherein the additional extracellular matrix molecular component is a protein selected from the group consisting of other forms of fibrillar collagen, elastin, and fibronectin.
35. The method of 34, wherein the type I, II, III, V collagen, other form of fibrillar collagen, elastin, and/or fibronectin are obtained from a different species than the human or other mammalian subject.
36. The method of 34, wherein the type I, II, III, V collagen, other form of fibrillar collagen, elastin, and/or fibronectin are obtained from stem cells isolated from the subject.
37. The method of any of 28-36, wherein the damaged connective tissue comprises a wound, broken or fractured bone, ruptured tendon, hernia, damaged barrier membrane, or inflammation of a connective tissue.
38. The method of any of 28-37, wherein the method speeds repair of the damaged connective tissue.
39. A method of altering a chemical or physical property of the liquid crystalline collagen composition of any one of 1-9, the method comprising: (i) adding the composition to a scaffold; (ii) stacking rolling, or bending the composition; or (iii) mineralizing the composition with calcium phosphate.
40. A method of growing cells in culture, the method comprising adding the liquid crystalline collagen composition of any one of 1-9 to a cell culture, whereby cells in the culture become bound to or aligned with fibril-like structures in the composition.
41. A medical device comprising the liquid crystalline collagen composition of any one of 1-9
42. A tissue scaffold comprising the liquid crystalline collagen composition of any one of 1-9.
43. The tissue scaffold of 42, further comprising a plurality of cells, preferably stem cells, and more preferably mesenchymal stem cells.
44. The composition of 4, wherein the fibril-like structures further comprise

contrasting layered bands having a layer tilt angle of ˜45° compared to a longitudinal axis of the fibril-like structures, the bands comprising chevron-like defect structures and a chevron-like interface in a center of the structures.

### BRIEF DESCRIPTION OF THE DRAWINGS

FIGS. 1A-1H show collagen fibers produced in vitro by a prior art flow-induced crystallization method. On the left, a series of four images from a movie (FIGS. 1A-1D) show the repair of a broken end of an extended collagen fiber (white arrow) by its reintroduction into and extrusion from a collagen solution. At the top center is an optical image (FIG. 1E) showing flow-induced crystallization fiber organization. At the top right is a scanning electron micrograph (SEM) (FIG. 1F) of a fiber pulled from a solution containing collagen/decorin. The bottom center panel (FIG. 1G) shows a transmission electron micrograph (TEM) of highly-aligned collagen fibrils in a flow induced crystallization fiber, and the lower right panel (FIG. 1H) shows the area from which the TEM of FIG. 1G was enlarged.

FIGS. 2A-2E show stable liquid crystal collagen, or SLCC in a stable, highly-densified, liquid crystal collagenous sheet. The top left (FIG. 2A) and top center (FIG. 2B) panels show TEMs of local fibril orientation in one “array” of fibrils with all collagen monomers in alignment (arrows show direction). Note the banding pattern. A photo of actual stable collagen sheet is shown in the photo inset in FIG. 2A. The center left (FIG. 2C) panel shows TEM of fibril arrays that meet and change direction. The center panel (FIG. 2D) shows a polarization image of the “crimp” that develops. The far-right panel (FIG. 2E) is a low magnification polarization microscopy image that shows long range organization.

FIG. 3 shows metastable liquid crystal collagen (MLCC) in a lower stability collagen sheet. The left panel (FIG. 3A) shows a TEM micrograph of the collagen fibril orientation in a single array (arrow shows alignment direction). Note that there is no characteristic D-banding, indicating lower stability (higher energy state). A photo of the actual lower stability collagen sheet is shown in the inset to FIG. 3A. The right panel (FIG. 3B) shows a burst release profile for the lower stability collagen sheet.

FIG. 4A shows a schematic representation of a method of treating a connective tissue injury by injecting an SLCC or MLCC composition of the present technology into the site of the injury, followed by application of extensional strain, physical therapy, and/or electrical stimulation of nearby muscle. FIG. 4B shows a schematic representation of a process of treating a collagen injury, such as a ruptured or injured tendon, by applying a bi-stable active collagen (AC) patch containing SLCC (slow release) and MLCC (faster release) collagen sheets to form a wrap around the tendon defect or injury. Collagen is released at two different rates into the injury.

FIGS. 5A-5C present a comparison of the mechanical properties of MLCC and SLCC30 materials based on direction of uniaxial loading.

FIG. 6 shows a differential interference contrast micrograph of a thick-section, cross-sectional cut of the MLCC material embedded in transmission electron microscopy resin. The light and dark bands, which correspond to fibril orientations, do not run perpendicular to the top and bottom surfaces of the material, but instead form vertical chevron-like defect structures.

FIGS. 7A-7C present a comparison of mechanical properties of scaffolds as a function of applied ultrasonic treatment duration. MLCC materials received no ultrasonic treatment. SLCC5, SLCC10, and SLCC30 received 5, 10, and 30 minutes of ultrasound, twice per day during the dehydration phase of the fabrication protocol.

FIGS. 8A-8B show acellular translocation of labelled active collagen monomer into a loaded, collagenase-treated collagen fiber. The image on the far-left (FIG. 8A) shows fluorescent collagen molecules added to a solution around a damaged fiber. The image on the right (FIG. 8B) shows that, over time, collagen monomers migrate into the loaded fiber.

FIGS. 9A-9D show schematic representations of tissue models with partial transection of a region of collagen fibers. At the top, in FIG. 9A, a central defect in DDCS (dense disorganized collagen strip) is shown. At second from top, in FIG. 9B, struts on either side of defect will be overloaded. Shown second from bottom, in FIG. 9C, and shown at the bottom, in FIG. 9D, are RTT (rat tail tendon) with a central defect and under load as well.

FIG. 10 shows a comparison of MLCC and SLCC linear modulus. SLCC exhibits a 4-fold higher modulus than MLCC (2.37+/−0.467 MPa compared to 0.586+/−0.083 MPa, n=7 for each sample).

FIG. 11 shows a representative SLCC stress-strain curve. Linear Modulus is determined in the linear part of the curve.

FIG. 12 shows a representative MLCC stress-strain curve. Both SLCC and MLCC exhibit ductile mechanical properties.

FIG. 13 shows cumulative tropocollagen delivery from MLCC and SLCC materials. Active collagen (AC) concentration is shown on the Y-axis.

FIG. 14 shows cumulative tropocollagen delivery from MLCC and SLCC materials reported as fraction of total protein content.

FIG. 15 shows elution rates per day for MLCC and SLCC materials.

FIG. 16 shows tropocollagen elution rate from MLCC material as an injectable formulation.

FIG. 17 shows a TEM image of SLCC material highlighting the fibril diameter and D-periodic banding structure. Note the change in fibril orientation.

FIG. 18 shows a TEM image of SLCC material.

FIG. 19 shows a TEM image of SLCC material showing the fibril dimensions and D-periodic banding structure.

FIG. 20 shows a TEM image of MLCC subjected to ultrasonic treatments (in the absence of “osmotic breathing” treatments). A 500 nm reference scale bar is shown at the bottom right corner of the image. Note that fibrils have ˜67 nm banding periodicity but exhibit less alignment at these short-range length scales than SLCC materials.

FIG. 21 shows an optical DIC image of SLCC material at 600× magnification. The scale bar=10 microns.

FIG. 22 shows an optical DIC image of SLCC after the material has been strained-to-failure. Note the enhanced resolution of fibrillar texture and alignment. The scale bar=10 microns.

FIG. 23 shows an optical DIC image of SLCC. The material has been cut perpendicular to the fibril-alignment axis, revealing the fibrillar texture and characteristics of the material. The scale bar=10 microns.

FIG. 24 shows a DIC of SLCC material showing the birefringent texture. The scale bar=50 microns.

FIG. 25 shows a DIC mosaic image showing the long-range propagation of the birefringent texture.

FIG. 26 shows a polarizing DIC mosaic of MLCC material showing the long-range propagation of the birefringent texture.

FIG. 27 shows an optical DIC image of MLCC material showing a bifurcation in birefringent texture. This morphological characteristic is common in liquid crystals. The scale bar=50 microns.

FIG. 28 shows an optical DIC of MLCC material. The scale bar=50 microns.

FIG. 29 shows an optical DIC of MLCC material cut perpendicular to the fibril-alignment axis. The resulting “sawtooth edge” is indicative of the underlying crimp-like morphology. The scale bar=50 microns.

FIG. 30 shows a TEM image of MLCC material. Note the fibril diameter and lack of D-periodicity. The scale bar at bottom=500 nanometers.

FIG. 31 shows a high-magnification TEM image of MLCC material. The scale bar at bottom=500 nanometers.

FIGS. 32A-32D show results of experimental repair of rat Achilles tendon using a bistable patch implant. FIG. 32A shows spring constants for paired experiments (treated vs. untreated), indicating a large increase in stiffness in treated animals at two weeks after implantation. FIG. 32B shows a histology series of a repair site (from left to right, hematoxylin & eosin (H&E) staining, Masson's trichrome staining, and picrosirius red staining) showing the punch defect site, the bistable collagen patch implant, intact collagen, and regions of collagen invasion and aligned new collagen. FIG. 32C shows high magnification images of the bistable patch region with cell migration into the patch region (left panel, H&E staining; right panel, Masson's trichrome). FIG. 32D shows a high magnification image of the bistable patch region with new collagen region (picrosirius red staining).

FIG. 33 shows a schematic representation of a method of producing a meniscus form of SLCC for use as an implant to treat a connective tissue injury.

### DETAILED DESCRIPTION

Efforts to produce 3D fibrillar collagen scaffolds with biomimetic densities and organizational motifs for tissue engineering and regenerative medicine have previously resulted in limited success. Scaffolds obtained by previous methods often exhibit organizational motifs that persist over very small local distances or fail to maintain characteristic fibril morphologies of native tissue (80-200 nm diameter fibrils with ˜67 nm periodic banding). The approach of the present technology is to utilize a biomimetic crowding/confining mechanism to densify and organize the collagen substrate in concert with ultrasound application and fluid-cycling to control fibril morphological characteristics.

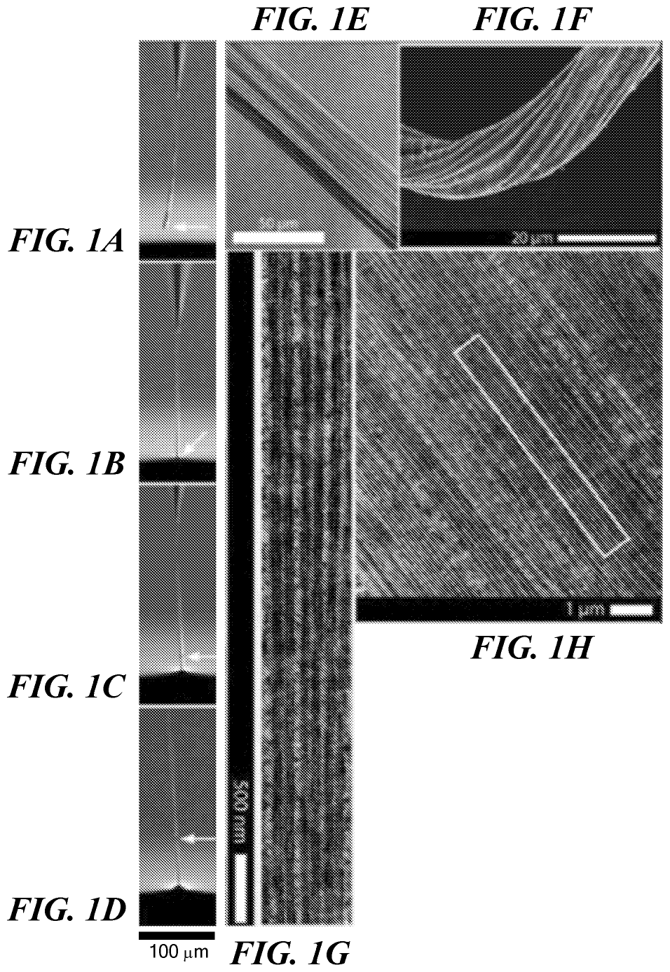

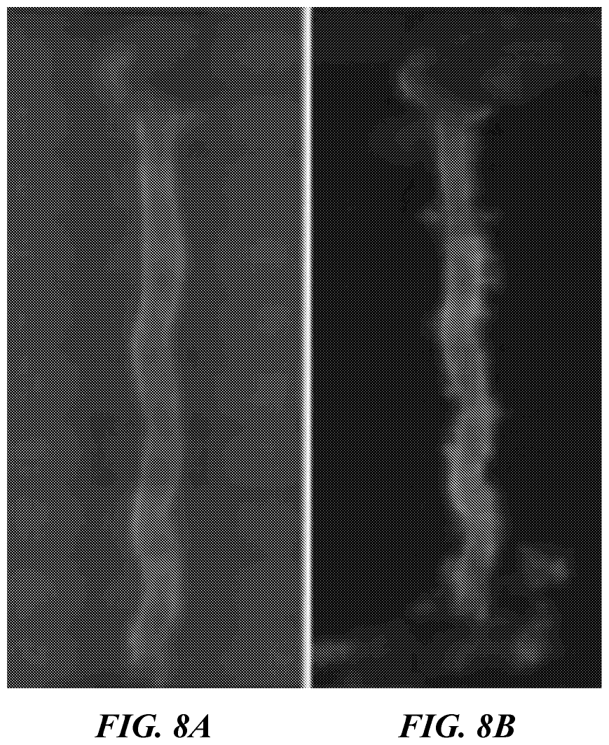

The low level of tropocollagen at the site of injury is a major limiting factor in the repair process. Assembly of collagen has been characterized in the literature. Collagens and their complementary associated extracellular matrix, ECM, molecules (i.e., proteoglycans, MMPs, fibronectin, etc.), are part of an autonomous structural system which has the capacity to self-assemble into load-bearing elements, spontaneously, in the path of force, precisely where they are needed. The assembly of tropocollagen into load-bearing tissues at regions of high loading is a fundamental property of modern metazoans. Mechanical strains are known to drive structure formation in other ECM biopolymers (e.g. fibronectin) and to promote crystallization in synthetic polymer systems. Recent studies have shown that collagen, in particular, will both form and retain structures (e.g. fibrils) directly in the path of tensile strains (see FIGS. 1A-1H, 8A-8B, and Paten et al.). Stopak et al. have shown that tropocollagen can be incorporated into prospective tendon, and a collagen patch comprising atelocollagen has improved rotator cuff repair in a rabbit model. In many cases, the supply of collagen is limited and impairs the ability to repair damaged tissue. In the present technology, collagen is supplied to damaged tendons under the appropriate chemical, mechanical, and geometric conditions in order to promote a rapid, high quality repair. The present technology includes a collagen delivery system that uses implanted SLCCS and/or MLCCS at a repair site, which can be used to deliver collagen, such as collagen monomer, to an injury site to repair tendons ex vivo or in vivo.

The present technology provides the ability to use a collagenous implant to act as a delivery vehicle for active collagen monomer. As part of the regular wound healing cascade, damaged or diminished collagen fibrils in the wound site are repaired by de novo collagen monomer fabricated in the local cell population. However, due to the paucicellular nature of tendon, there is a significant lack of monomeric collagen production in the wound site. Damaged fibrils in the tissue are therefore slow to repair. The materials and methods of the present technology offer the ability to sustain delivery of collagen monomers over an extended period of time. Delivering supplemental collagen monomer into the wound in a controlled manner is thus a promising therapeutic approach.

The present technology provides a Stable Liquid Crystal Collagen Scaffold (SLCCS) cohesive network of collagen fibrils organized into a chiral smectic liquid crystal array (and with potential for other LCC organizational motifs). The fibrils are banded and aligned, and the fibril arrays change orientation by +/−70 degrees at ˜20 micron intervals; these parameters can vary by +/−5%, 10, or 20%. The collagen fibril network is produced via inducing liquid crystal phasing of collagen and dehydrating before condensation of monomers into densely-packed fibrils. The fibril network contains pores which can be used to incorporate therapeutics/cell lines. The technology provides tunable fibril and pore characteristics via promoting/preventing nucleation and growth phases of fibril precipitation. Altering these characteristics can alter delivery rate of monomer, porosity, and scaffold mechanical properties.

Another aspect of the present technology is an SLCCS substrate or scaffold containing aligned, banded collagen fibrils whose orientation persists over the scale of centimeters. The fabrication of the substrate is easily scaled-up and can be used to build large substrates or tissue equivalents by stacking SLCCS sheets. Nanoscaled and microscaled pores present in the substrates are orders of magnitude smaller than those found in dehydrated collagen gels of the prior art, and their size can inhibit disadvantageous burst release of therapeutics contained in the pores.

The SLCCS collagen network and scaffold of the present technology offer tunable fibril characteristics and density, which permits the formulation of constructs that mimic particular native collagenous tissues. The scaffolds can be stacked or rolled to create collagenous tissue equivalents (both with and without cells). The collagen networks and scaffolds find application in cornea, bone, tendon, and ligament tissue engineering, and can be seeded with therapeutics or stem cells for applications in wound healing. They also can be used for in vitro laboratory studies, such as investigations of the effect of aligned collagen on stem cell behavior and the effect of collagen fibril alignment on the rate of drug delivery.

SLCCS scaffolds consist of >95% collagen type I fibrils condensed into a dense fibrillar sheet. SLCCSs are fabricated via inducing liquid crystal phasing of a dense collagen solution prior to initiating fibrillogenesis. The organizational motif resembles a chiral smectic liquid crystal, with fibrils aligned along their long-axis into fibril arrays. At ˜20 micron intervals, the orientation of fibril arrays reorients ˜70 degrees and returns to the baseline orientation after an additional 20 microns; these parameters can vary by +/−5%, 10, or 20%. This morphology resembles a chiral smectic liquid crystal and to our knowledge, collagen scaffolds with this organizational motif have never been reported in the literature. Most importantly, this organizational pattern persists uninterrupted over a length of at least 45 millimeters and represents a significant advancement in the development of collagen scaffolds with long-range order. Fibrils within the SLCCS recapitulate the nano- and micro-hierarchical organizations of native collagenous tissues, while maintaining characteristic morphologies of native fibrils. While the organization of SLCCS span over a considerable distance, the macroscale organization resembles a chiral smectic liquid crystal and does not recapitulate the macroscopic organization of native tissues.

SLCCSs have potential applications in a number of tissue engineering and regenerative medicine applications. SLCCS can be fabricated as a thin sheet of predictably organized collagen. These sheets can be used for laboratory research for investigators examining the effect of aligned collagen on cell behavior and diffusion of small molecules in anisotropic matrices. SLCCS sheets also can be stacked to create larger 3-dimensional structures resembling cornea, bone, tendon, and ligament for tissue engineering applications. Furthermore, SLCCS can serve as a scaffold for cell and therapeutic delivery in regenerative medicine.

Characteristics of SLCCS collagen networks and scaffolds, including collagen concentration, dimensions, fibril morphology, and pore size, can be adjusted via manipulating the parameters of the liquid crystal system, including length of time dehydrating in polyethylene glycol, presence of ionic species, concentration of ionic species, temperature, length of time in temperature, confining geometries, and application of mechanical stimulation. Further modifications to the SLCCS include addition of exogenous crosslinking, proteoglycans, non-collagenous proteins, glycosaminoglycans, and other fibrillar collagens (II,III,V,XI) to control mechanical and morphological properties of the scaffold.

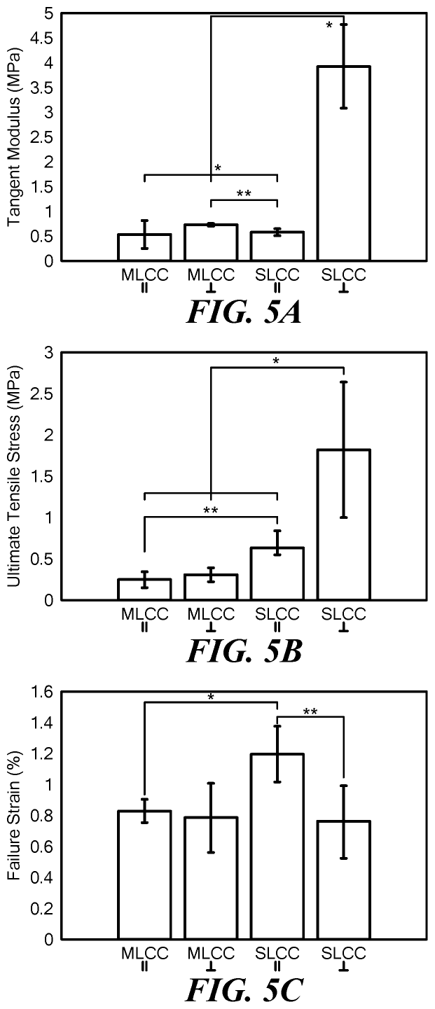

Another aspect of the SLCC materials is the mechanical anisotropy. FIGS. 5A-5C present a comparison of MLCC and SLCC30 materials based on direction on uniaxial loading. The “30” designation at the end of SLCC indicates 30 minutes of ultrasound treatment applied to the SLCC material. Tensile modulus is compared in FIG. 5A; the center of FIG. 5, (FIG. 5B), compares material strength; and at the bottom, FIG. 5C, failure strain is compared. Parallel bars correspond to tension applied parallel to the long-axis of the birefringent texture. Perpendicular bars correspond to tension applied perpendicular to optical texture. SLCC displays significant mechanical anisotropy and represents an improvement in modulus and strength relative to metastable materials. The single and double asterisks, * and **, each represent significant differences (p<0.05) between the indicated populations.

As demonstrated in FIG. 5A and FIG. 5B, the modulus and tensile strength are significantly higher when tested perpendicular to the birefringent texture (mean 3.92±0.84 MPa and 1.83±0.81 MPa, respectively) than the parallel direction (mean 0.58±0.07 MPa and 0.66±0.20 MPa, respectively). As demonstrated in FIG. 5C, SLCC scaffolds demonstrate significantly lower failure strains when tensioned perpendicular to the optical texture than the parallel (0.76±0.23 vs. 1.19±0.18). The MLCC materials exhibit no discernable mechanical anisotropy, possibly due to the metastable nature of the fibrous material and its high failure strain. However, MLCC materials have significantly less stiffness and tensile strength than SLCC materials, mean=0.73±0.03 MPa and 0.33±0.08 MPa, respectively, when tested perpendicular to the birefringent texture.

The present technology further provides a fabrication technique for producing Metastable Liquid Crystal Collagen Scaffolds (MLCCS) for the purpose of rapid delivery of large quantities of collagen monomer to collagenous defects. These scaffolds consist of >95% collagen type I fibril-like structures condensed into a dense fibrillar sheet. Most notably, the collagen fibrils exhibit a unique morphology, which is characterized by collagen fibrils that lack the characteristic D-periodicity of native fibrils.

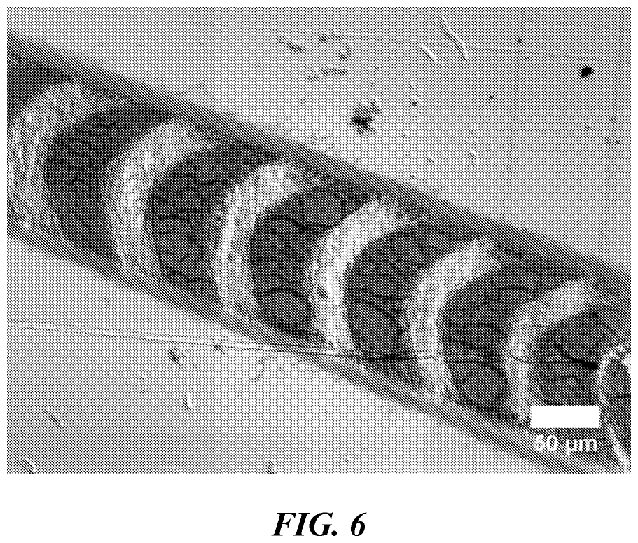

The MLCCS contains metastable liquid crystal collagen (MLCC) material with tunable stability. FIG. 6 shows a differential interference contrast micrograph of a thick-section, cross-sectional cut of the MLCC material embedded in transmission electron microscopy resin. The light and dark bands, which correspond to fibril orientations, do not run perpendicular to the top and bottom surfaces of the material, but instead form vertical chevron-like defect structures. An aspect of this material is the vertical chevron defect geometry. When cut in cross-section or longitudinal (any axis perpendicular to the planar axis of fibril orientation), vertically oriented chevron-like structures are observed (FIG. 6). These defects are characteristic of smectic C structures and arise from anchoring of the tropocollagen molecules at the surface layer (the molecular layer in contact with the dialysis membrane). As the smectic layers progress from the surface boundary into the bulk, the height of each layer in the smectic geometry decreases, resulting in the characteristic displacement of layers in the vertical chevron defect. At the chevron interface, which is usually at the center of the material, the layer tilt angle abruptly changes its sign. The layer tilt angle is generally found to be of the same order as the molecular tilt angle. The materials disclosed here feature a chevron interface in the center of the material and have a layer tilt angle approximately equivalent to the molecular tilt angle (˜45°).

This periodic banding pattern of a fibril is a distinguishing feature of fibrils and arises from the precise lateral arrangement of molecules within a fibril. A lack of D-periodicity suggests that monomers residing in the MLCCS fibrils are not situated into proper positional configurations and are therefore more susceptible to dissociation from the fibrillar scaffold. Once dissociated, monomer will self-assemble into new and existing fibrils of the host tissue thus accelerating wound repair. MLCCSs are extremely dense and have the potential to deliver monomeric collagen to a wound site over a period of weeks to months.

The present approach is a departure from current therapeutic strategies for the repair of collagenous defects. MLCCS compositions can be used as a standalone implant or in conjunction with other available collagen products. A scaffold containing MLCCS features tunable delivery rates of collagen monomer to make it suitable for a range of defects and therapies. The scaffold also features nanometer- and/or micrometer-size (diameter) pores capable of transporting additional therapeutics or human cell lines.

MLCCS collagen networks are cohesive fibrous networks of aligned collagen fibrils which lack characteristic 67 nm D-periodicity. The fibrils exist in arrays that exhibit undulating morphology instead of typical arrays of parallel, linear fibrils. The high density undulating fibrils leave pores in network for therapeutics. The fibril networks are produced via inducing liquid crystal phasing of collagen, with dehydrating before condensation of monomers into fibrils. The fibril and pore characteristics are tunable via promoting or preventing nucleation and growth phases of fibril precipitation. Altering these characteristics alters delivery rate of monomer, porosity, and scaffold mechanical properties. The stability of MLCCS can be tuned through exposure to ultrasound, thermal cycling or crosslinking agents.

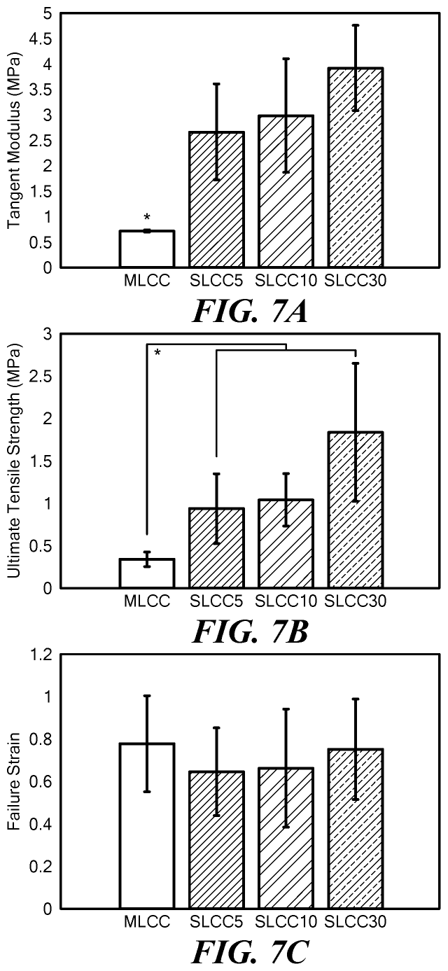

FIGS. 7A-7C present a comparison of mechanical properties of scaffolds as a function of applied ultrasonic treatment duration. MLCC materials received no ultrasonic treatment. SLCC5, SLCC10, and SLCC30 received 5, 10, and 30 minutes of ultrasound, twice per day during the dehydration phase of the fabrication protocol. In the top panel, FIG. 7A, tensile modulus is characterized. In the center panel, FIG. 7B, material strength is characterized, and failure strain is characterized in FIG. 7C. In all cases, mechanical testing was applied perpendicular to the birefringent optical texture (the strongest material axis). The asterisks, *, represent p<0.05 for indicated populations.

Another aspect of this material is the tunable mechanical properties as a function of applied ultrasound durations. Modulating the duration of ultrasound during the dehydration phase of assembly from 5, 10, and 30 minutes results in an increase in the mechanical integrity of collagen scaffolds. In all cases, tangent modulus significantly increases relative to MLCC materials (mean 0.73±0.02 MPa) with applied ultrasound as shown in FIG. 7A. No detectable significant difference is found between conditions with 5, 10, or 30 minutes of applied ultrasonic treatment (2.66±0.95, 2.99±1.12, and 3.92±0.84 MPa, respectively). As demonstrated in FIG. 7B, material strength of SLCC10 and SLCC30 is significantly higher than MLCCs (1.04±0.31 and 1.84±0.81 vs. 0.34±0.08 MPa). There is no significant difference between the ultimate strength of SLCC5 (0.94±0.41 MPa) and MLCC materials. No difference was determined in the failure strain of any mechanically tested scaffolds, as shown in FIG. 7C (0.78±0.03, 0.65±0.21, 0.67±0.28, and 0.75±0.23 for MLCC, SLCC5, SLCC10, SLCC30 scaffolds, respectively).

MLCC collagen fibrils have a higher free energy than dehydrated collagen gels used in current therapeutics and can deliver monomer at a faster rate. An MLCCS construct has a significantly higher density and lower stability than dehydrated collagen gels used in current market products. MLCC collagen networks and scaffolds thus contain a larger reservoir of collagen monomer that makes them ideal for delivering collagen monomer to host tissue over a period of time up to months. Pores of MLCC compositions are orders of magnitude smaller than dehydrated collagen gels of the prior art and will inhibit disadvantageous burst release of therapeutics. Their tunable pore size and density permits formulations of constructs for different therapeutics and/or cell types. Their tunable fibril characteristics and density permits formulations containing constructs that are tissue-specific and have different release characteristics.

Monomers can dissociate from the MLCCS fibrous network at a higher rate than the relatively stable monomers in previously available collagenous therapeutics containing native-like banded collagen. Once solubilized, monomer settles in its lowest free energy state and thus accumulates in the loaded native fibrils of the wound site, resulting in decreased time to healing. Monomers can also assemble along the path of the tissue strain to create de novo fibers.

Characteristics of the scaffold including concentration, fibril morphology, and pore size can be adjusted to alter delivery rate and profile of both collagen and additional therapeutics. This can be achieved via manipulating the parameters of the liquid crystal system, including length of time dehydrating in polyethylene glycol, presence of ionic species, concentration of ionic species, temperature, length of time in temperature, confining geometries, osmotic cycling, and application of mechanical stimulation. Potential modifications to the MLCC include addition of exogenous crosslinking, proteoglycans, glycosaminoglycans, non-collagenous proteins, and other fibrillar collagens (II,III,V,XI) to control mechanical and morphological properties of the scaffold.

Both SLCCS and MLCCS compositions can be used to treat injuries to tendon, ligament, and joint capsules, for example. Tendon injuries that can be treated include, for example, shoulder rotator cuff tendons, Achilles tendon, and patellar tendon.

Methods of producing SLCCS and MLCCS are further described below.

In one aspect of the technology, SLCCS can be created by a process termed “osmotic breathing”. In an embodiment of the process, a collagen solution is dehydrated by dialysis for 12 hours at 4° C. against acidic, with a pH of about 1.5 to 2.5, 10% polyethylene glycol (MW˜35000 Daltons) dissolved in deionized water. In some embodiments, the collagen solution is concentrated to about 10-15 mg/mL.

The collagen solution is then placed into a fresh dialysis bag which is placed in acidic 40% polyethylene glycol dissolved in deionized water. The collagen solutions are subjected to ultrasound for 30 minutes, twice per day, over the course of 7 days. The dialysis bags are then transferred to 40% polyethylene glycol dissolved in 1×PBS at 7.4 pH at 35° C. and then to 1×PBS solution at 35° C. at 24-hour intervals over the course of 7 days. In some embodiments, the sonication is at about 130 watts power and about 20 kHz. In some embodiments, ultrasound is necessary to stabilize tropocollagen into natively banded fibrils.

Another aspect of the technology is a process of creating MLCCS by osmotic breathing. An embodiment of the process includes first dehydrating a collagen solution by dialysis against acidic (pH about 1.5-2.5) 10% polyethylene glycol (MW˜35000 Daltons) dissolved in deionized water for 12 hours at 4° C. The solution is then placed into a fresh dialysis bag which is placed into acidic 40% polyethylene glycol dissolved in deionized water for 7 days at 4° C. The dialysis bag is then transferred to neutralized 40% polyethylene glycol dissolved in 1×PBS for 4 days at 4° C. The PEG/collagen solution is then warmed to 37° C. to initiate fibrillogenesis for 48 hours.

Yet another aspect of the technology is a process of assembling a combined MLCC/SLCC bi-stable patch by polymerizing a dense (e.g., 15 mg/mL) solution of neutralized collagen between the MLCC and SLCC sheets and subjecting the patch to neutral 40% PEG-1×PBS at 37° C. for 24-hours. Optionally, cross-linking can then be utilized (such as by UVA or another known method), and SLCCs are cross-linked by exposure to UV and/or riboflavin per a modified Dresden protocol. An original cross-linking procedure, commonly referred to as the Dresden protocol, calls for UVA exposure for 30 minutes at 3 mW/cm⁻². UV exposure time from 10 to 40 minutes is used to achieve a range of cross-linking densities as assessed by denaturation temperature. In some embodiments, for each class of material produced, sheets are cut such that long-axis is perpendicular to the birefringent texture (parallel to fibril orientation).

In a further process of the technology, the release rates of MLCC are tuned by altering the duration of ultrasound (US) and/or other conditions. In some embodiments, MLCC and SLCC are implanted or injected (after being fragmented) to serve as a tissue filler in a subject. In some embodiments, ultrasound is applied to the subject to convert the MLCC to SLCC and fuse the particles into a more solid, degradation-resistant state that possesses an overall lower energy.

In some embodiments, MLCCS contain metastable liquid crystal collagen (MLCC) material with tunable stability. As shown in FIG. 6, the light and dark bands, which correspond to fibril orientations, do not run perpendicular to the top and bottom surfaces of the material, but instead form vertical chevron-like defect structures. In some embodiments, an aspect of this MLCC material is the vertical chevron defect geometry. When cut in cross-section or longitudinal (any axis perpendicular to the planar axis of fibril orientation), vertically oriented chevron-like structures are observed (FIG. 6). In some embodiments, these defects are characteristic of smectic C structures and arise from anchoring of the tropocollagen molecules at the surface layer (the molecular layer in contact with the dialysis membrane). As the smectic layers progress from the surface boundary into the bulk, the height of each layer in the smectic geometry decreases, resulting in the characteristic displacement of layers in the vertical chevron defect. At the chevron interface, which is usually at the center of the material, the layer tilt angle abruptly changes its sign. In some embodiments, the layer tilt angle is generally found to be of the same order as the molecular tilt angle. In some embodiments, the MLCC materials disclosed here feature a chevron interface in the center of the material and have a layer tilt angle approximately equivalent to the molecular tilt angle (˜45°).

Any of the materials of the present technology can be employed in methods of promoting or accelerating healing of damaged connective tissue. The damaged connective tissue can include a wound, such as a wound caused by trauma or infection, a broken or fractured bone, a ruptured tendon, a hernia, or inflammation in a connective tissue. The healing or repair can be undertaken by surgical implantation or injection of the material at or near the damaged site. The healing or repair also can include surgical, biochemical, pharmacological, genetic, physical, stem cell, or other treatment of the damaged connective tissue and/or other parts of the subject's body. Physical methods of treatment can include one or more of continuous passive motion, ultrasound, vibration, and electromyostimulation. The material can be provided as SLCCS, MLCCS, or a bistable patch containing SLCC and MLCC. The collagen in the material can be type I, II, III, or V collagen, or another form of fibrillar collagen. The collagen can be isolated from cells of the subject, isolated collagen from the same species as the subject, or isolated collagen from a different species. The material can contain one or more additional extracellular matrix molecular components, such as elastin or fibronectin, from the cells of the subject, from the same species as the subject, or from a different species.

Methods to aid in repairing damaged connective tissue as described above can accelerate healing of a damaged connective tissue, or reduce the time to full load bearing capacity after damage to connective tissue, or increase the stiffness or load bearing of a connective tissue at a given time after an injury. Such methods can be implemented in a human or other mammalian subject. The methods can include the following steps: (a) providing any of the liquid crystalline collagen compositions described herein; and (b) placing the composition into a region that contains damaged connective tissue in the subject. Following step (b), the damaged connective tissue is at least partially repaired or healed, or stiffness, or spring constant, or load bearing capacity of the connective tissue is increased. Step (a) of the method can include the following: (a1) obtaining cells from the subject; (a2) culturing the cells and optionally inducing differentiation and/or proliferation of the cells and/or collagen secretion by the cells; (a3) harvesting collagen from the culture; and (a4) forming any of the liquid crystalline collagen compositions described herein using the harvested collagen. Step (b) can be performed continuously or intermittently over a period of time.

### EXAMPLES

### Example 1. Formation of Stable Liquid Crystalline Collagen Scaffolds

Stable Liquid Crystal Collagen Scaffolds (SLCCS) were created by dehydrating 10 mL of telopeptide intact bovine collagen (TELOCOL, Advanced BioMatrix) against acidic 10% polyethylene glycol dissolved in deionized water (35,000 MW) for 12 hours at 4° C. The solution was extracted and placed in a fresh 45 mm×15 mm dialysis bag (3500MWCO) and then placed in acidic 40% polyethylene glycol dissolved in deionized water. These samples were subjected to ultrasound using a 3510 Branson sonicator for 30 minutes, twice per day over the course of 7 days. Samples were then transferred to 40% polyethylene glycol dissolved in 1×PBS at 7.4 pH at 35° C. and then to 1×PBS solution at 35° C. at 24-hour intervals over the course of 7 days. This process is termed “osmotic breathing”. The resulting scaffold contained 31 mg of type I collagen with dimensions of 45 mm×15 mm×50 microns and featured a fingerprint pattern under polarized light that is characteristic of liquid crystalline order. This fingerprint pattern persisted over >90% of the scaffold and corresponds to shifts in the alignment of fibril arrays (resembling a chiral smectic liquid crystal) in the X-Y plane.

The density of this material was about 800 mg/cm³. Fibrils were 80-100 microns in diameter and organized into aligned fibril arrays aligned along the long-axis of fibril. Fibrils had an ˜85 nm periodic banding structure that closely resembles, but does not duplicate, the D-periodicity of native collagen fibrils. This suggests that monomer in reconstituted fibrils were settled in stable 3-dimensional configurations.

### Example 2. Formation of Metastable Liquid Crystalline Collagen Scaffolds

Metastable Liquid Crystal Collagen Scaffolds (MLCCS) were created by dehydrating 10 mL of telopeptide intact bovine collagen (TELOCOL, Advanced BioMatrix) against acidic 10% polyethylene glycol dissolved in deionized water (35,000 MW) for 12 hours at 4° C. The solution was extracted and placed in a fresh 45 mm×15 mm dialysis bag (3500 MWCO) and then placed in acidic 40% polyethylene glycol dissolved in deionized water for 7 days at 4° C. The dialysis bag was then transferred to neutralized 40% polyethylene glycol dissolved in 1×PBS for 4 days at 4° C. The PEG/collagen solution was then warmed to 37° C. to initiate fibrillogenesis for 48 hours. The resulting scaffold contained 30 mg of type I collagen with dimensions of 45 mm×15 mm×100 microns and featured a fingerprint pattern under polarized light that is characteristic of liquid crystalline order. This fingerprint pattern persisted over >90% of the scaffold and corresponds to shifts in the alignment of fibril arrays in the X-Y plane.

The density of this material was about 200-500 mg/cm³. Fibrils were 80-100 microns in diameter and organized into undulating fibril arrays aligned along the long-axis of fibril. They also lacked the characteristic D-periodicity banding of native fibrils. This morphology is atypical in native tissues and suggests that monomers in reconstituted fibrils are settled in improper 3-dimensional configurations of a local energy minimum that is less stable than the SLCCS.

MLCC material were cross-sectioned, and the cut MLCC material was embedded in transmission electron microscopy resin. Surprisingly in FIG. 6, the light and dark bands, which correspond to fibril orientations, were not found to run perpendicular to the top and bottom surfaces of the material, but instead form vertical chevron-like defect structures. When the MLCC material was cut in cross-section or longitudinal (any axis perpendicular to the planar axis of fibril orientation), vertically oriented chevron-like structures were observed (FIG. 6). The materials were found to have a chevron interface in the center of the material and have a layer tilt angle approximately equivalent to the molecular tilt angle (˜45°). The MLCCS were found to contain metastable liquid crystal collagen (MLCC) material with tunable stability.

### Example 3. Characterization of SLCCS and MLCCS Mechanical Strength and Release Rate

SLCC and MLCC materials were prepared as described in the above examples and compared. Mechanical specimens were prepared by cutting sheets are cut into 10 mm (5 mm max and 3 mm gauge width) dog-bone tensile test specimens. The strips were cut such that long-axis is perpendicular to the birefringent texture (parallel to fibril orientation).

Mechanical testing was performed within 6 hours post-fabrication, specimens were mounted between spring-loaded grips and preloaded to 0.002N in a custom mechano-bioreactor. All specimens were strained to failure at a rate of 6 mm/min while recording the load. Linear modulus (Pa), onset of plastic deformation (mm), ultimate tensile stress (Pa), and toughness (J) were determined.

FIGS. 5A-5C present a comparison of MLCC and SLCC30 materials based on direction on uniaxial loading, the “30” designation at the end of SLCC indicates 30 minutes of ultrasound treatment applied to the SLCC material. FIG. 5A compares tensile modulus; FIG. 5B compares material strength; and at the bottom, FIG. 5C, failure strain is compared. Parallel bars correspond to tension applied parallel to the long-axis of the birefringent texture. Perpendicular bars correspond to tension applied perpendicular to optical texture. SLCC displayed significant mechanical anisotropy and represent an improvement in modulus and strength relative to metastable materials. The single and double asterisks, * and **, each represent significant differences (p<0.05) between the indicated populations.

As demonstrated in FIG. 5A, top panel and FIG. 5B, center panel, the modulus and tensile strength were significantly higher when tested perpendicular to the birefringent texture (mean 3.92±0.84 MPa and 1.83±0.81 MPa, respectively) than the parallel direction (mean 0.58±0.07 MPa and 0.66±0.20 MPa, respectively). As demonstrated in FIG. 5C, bottom panel, SLCC scaffolds exhibited significantly lower failure strains when tensioned perpendicular to the optical texture than the parallel (0.76±0.23 vs. 1.19±0.18). The MLCC materials exhibited no discernable mechanical anisotropy, possibly due to the metastable nature of the fibrous material and its high failure strain. However, MLCC materials had significantly less stiffness and tensile strength than SLCC materials, mean=0.73±0.03 MPa and 0.33±0.08 MPa, respectively, when tested perpendicular to the birefringent texture.

Ultrasound duration was then varied and tested. MLCC materials received no ultrasonic treatment. SLCCS, SLCC10, and SLCC30 received 5, 10, and 30 minutes of ultrasound, twice per day during the dehydration phase of the fabrication protocol. In FIG. 7A, tensile modulus is characterized. In the center panel (FIG. 7B), material strength is characterized, and failure strain, (FIG. 7C), is characterized in the bottom. In all cases, mechanical testing was applied perpendicular to the birefringent optical texture (the strongest material axis). The asterisks, *, represent p<0.05 for indicated populations.

Modulating the duration of ultrasound during the dehydration phase of assembly from 5, 10, and 30 minutes resulted in an increase in the mechanical integrity of collagen scaffolds. In all cases, tangent modulus significantly increased relative to MLCC materials (mean 0.73±0.02 MPa) with applied ultrasound as shown in FIG. 7A. No detectable significant difference was found between conditions with 5, 10, or 30 minutes of applied ultrasonic treatment (2.66±0.95, 2.99±1.12, and 3.92±0.84 MPa, respectively). As demonstrated in FIG. 7B, material strength of SLCC10 and SLCC30 were significantly higher than MLCCs (1.04±0.31 and 1.84±0.81 vs. 0.34±0.08 MPa). There was no significant difference between the ultimate strength of SLCC5 (0.94±0.41 MPa) and MLCC materials. No difference was determined in the failure strain of any mechanically tested scaffolds, as shown in FIG. 7C (0.78±0.03, 0.65±0.21, 0.67±0.28, and 0.75±0.23 for MLCC, SLCC5, SLCC10, SLCC30 scaffolds, respectively).

Type I telocollagen is acetic acid extracted from young (8-12 wks) New Zealand white rabbit scleras (Pel-Freez Biologicals, Rogers, Ark.).

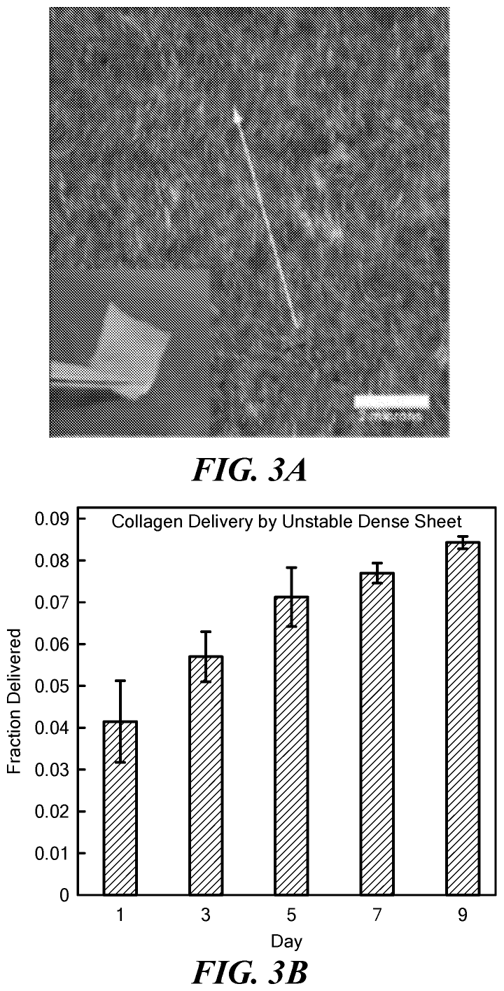

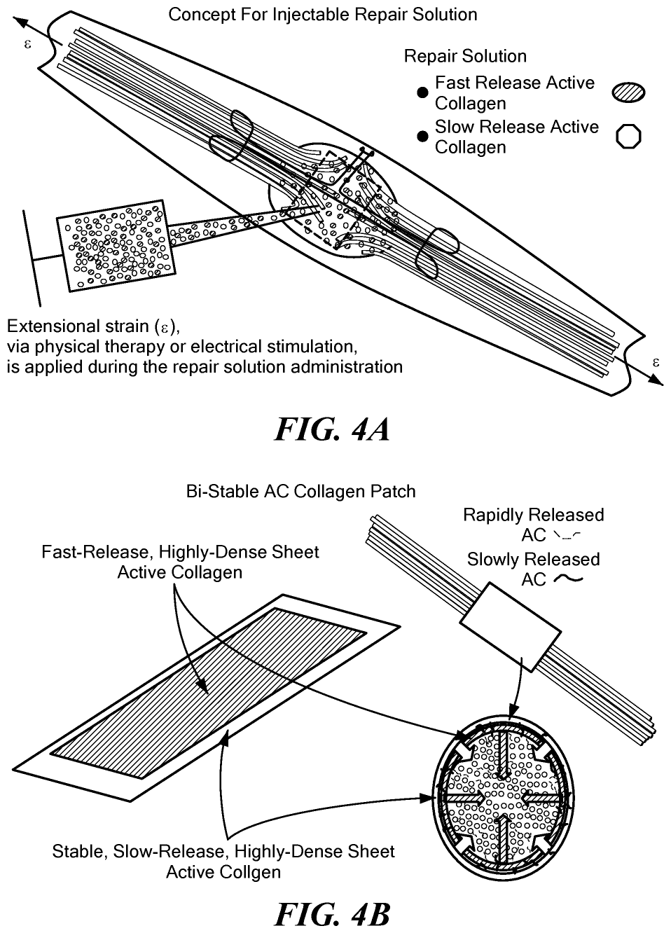

MLCCSs (as in FIGS. 3A-3B) are fabricated via molecular crowding to develop liquid crystal phasing of collagen mesogens through dialysis against acidic 40% polyethylene glycol (PEG). Once the mesogen concentration stabilizes (˜300 mg/mL) “metastable fibrillogenesis” is induced via exposure to PEG/1×PBS at pH=7.4.

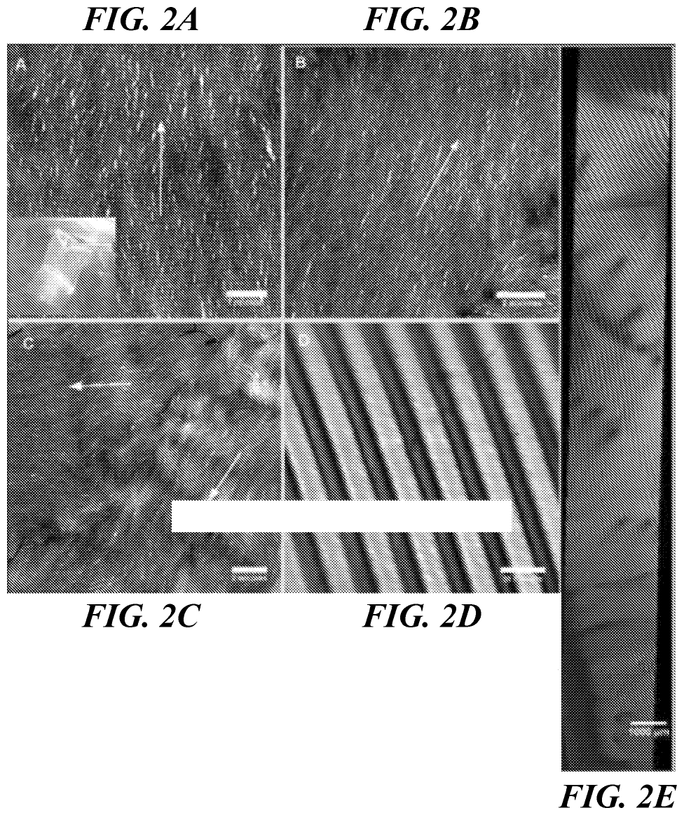

SLCCSs (as in FIGS. 2A-2E) are produced similarly but with two procedural changes: 1) During the initial dialysis, the densifying collagen solution is subjected to two 30-minute treatments per day of 130 W ultrasound (US) and 2) After 7 days, the sample is subjected to a 7-day PEG-PBS cyclic “osmotic breathing” treatment comprising alternating submersion into neutralized concentrated PEG-1×PBS and only 1×PBS at 37° C. at 24-hour intervals to induce “stable fibrillogenesis.”

Combined MLCC/SLCC bi-stable patch is assembled by polymerizing a dense (15 mg/mL) solution of neutralized collagen between the MLCC and SLCC sheets and subjecting the patch to neutral 40% PEG-1×PBS at 37° C. for 24-hours.

Modulating SLCC mechanics through UVA cross-linking: SLCCs are cross-linked by exposure to UV and riboflavin per a modified Dresden protocol. An original cross-linking procedure, commonly referred to as the Dresden protocol, calls for UVA exposure for 30 minutes at 3 mW/cm⁻². UV exposure time from 10 to 40 minutes is used to achieve a range of cross-linking densities as assessed by denaturation temperature.

Mechanical specimens: For each class of material produced, sheets are cut into 10 mm (5 mm max and 3 mm gauge width) dog-bone tensile test specimens. The strips are cut such that long-axis is perpendicular to the birefringent texture (parallel to fibril orientation).

Mechanical testing: Within 6 hours post-fabrication, specimens are mounted between spring-loaded grips and preloaded to 0.002N a custom mechano-bioreactor. All specimens are strained to failure at a rate of 6 mm/min while recording the load. Linear modulus (Pa), onset of plastic deformation (mm), ultimate tensile stress (Pa), and toughness (J) are determined.

Release rates of the SLCC, the MLCC and combined SLCC/MLCC bi-stable patches is determined by the following procedure: Monomeric collagen is labeled using standard methods for fluorescently labeling antibodies that result in an average of two fluorophores per tropocollagen. Labeled monomer is added to unlabeled monomer in a ratio of 1:1000 and then incorporated into each collagen sheet.

Determining kinetics of active collagen (AC) delivery: Each class of material is fabricated as described previously. Five samples are cut from each collagenous sheet (collagen content ˜7.75 mg) and submerged in physiologic salt buffer at 37° C. Release solution is collected and replaced at 24-hour intervals.

Tuning kinetics of MLCC-AC delivery: To tune the release rates of MLCCs, the duration of ultrasound (US) is altered. US is necessary to stabilize tropocollagen into natively banded fibrils. MLCCS samples are fabricated with fluorophore-labeled monomers as described above. Experimental conditions include a 1, 5, 10, 15, 20, or 25-minute treatment of 130 W US twice per day for seven days over the course of dehydration against acidic PEG. MLCC sheets are divided into 5 mm×5 mm squares. Release solution is collected and replaced at 24-hour intervals.

Quantifying AC release rates: Aliquots of release solution are examined via fluorescence imaging using a Nikon inverted microscope (ECLIPSE TE2000-E) equipped with a high speed EMCCD Camera (iXon Ultra 897, ANDOR). Fluorescence intensity of a solution is converted to collagen concentration by use of a standard relating fluorescence intensity to collagen concentration. After 14 days, all samples are dissolved in 0.5M HCl to determine the amount of undelivered tropocollagen. Fractional delivery (FDcoll) as a function of time is determined.

### Example 4. Demonstration of the Ability of Bi-Stable SLCC/MLCC Patches to Repair Tissue Defects In Vitro

Delivery of AC collagen produces a repair effect via MFSC on a simplified damaged tissue model and an excised, damaged native connective tissue in vitro. To demonstrate this process, collagen is delivered from a bi-stable collagen patch directly to models of damaged tissue that are subjected to mechanical stimulation. MFSC is demonstrated by participation of collagen from the patch in the repair of the defects in the tissue models. Two tissue models are used: 1) a dense disorganized collagen strip (DDCS), and 2) native rat tail tendon (RTT).

Collagenase tendinopathy model: To simulate tendinopathy, the tissue models are exposed to purified bacterial collagenase. Exposure time is standardized by tracking loss of constituent tropocollagen and measuring the change in tangent modulus.

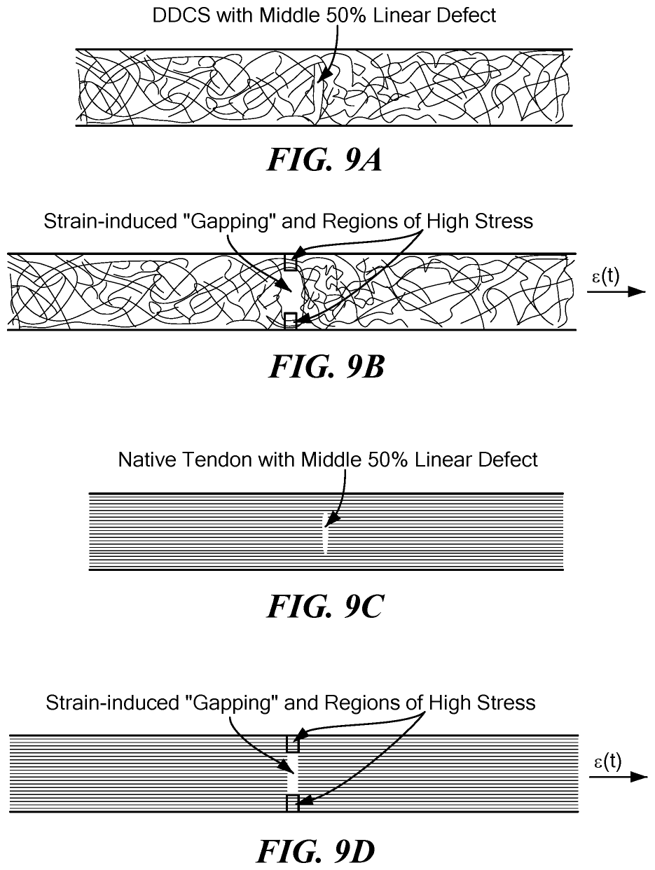

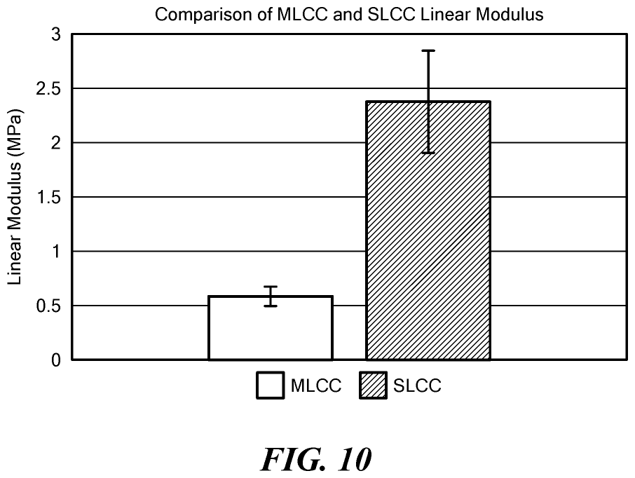

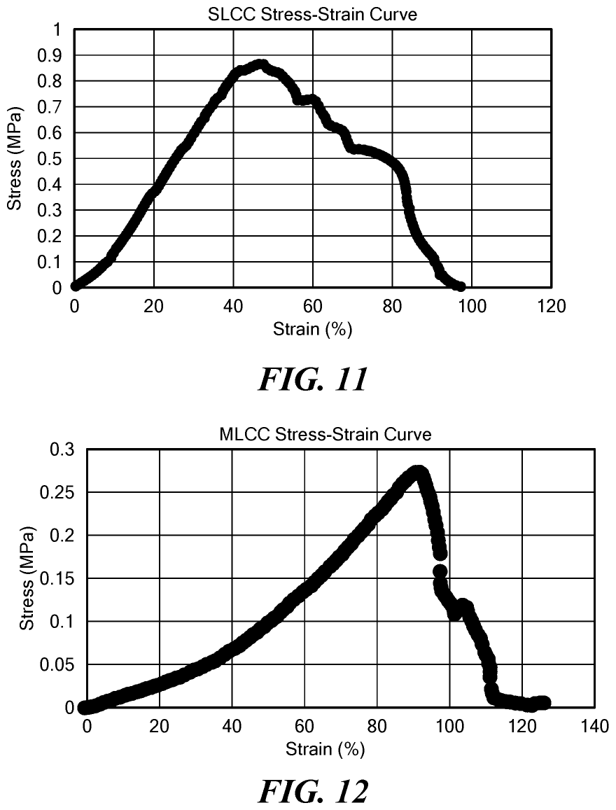

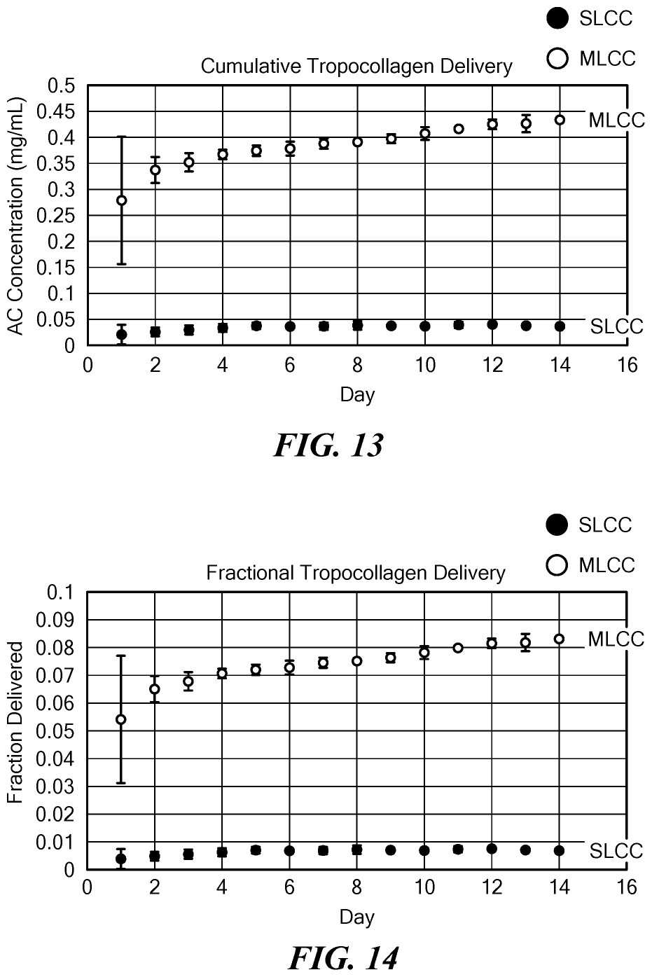

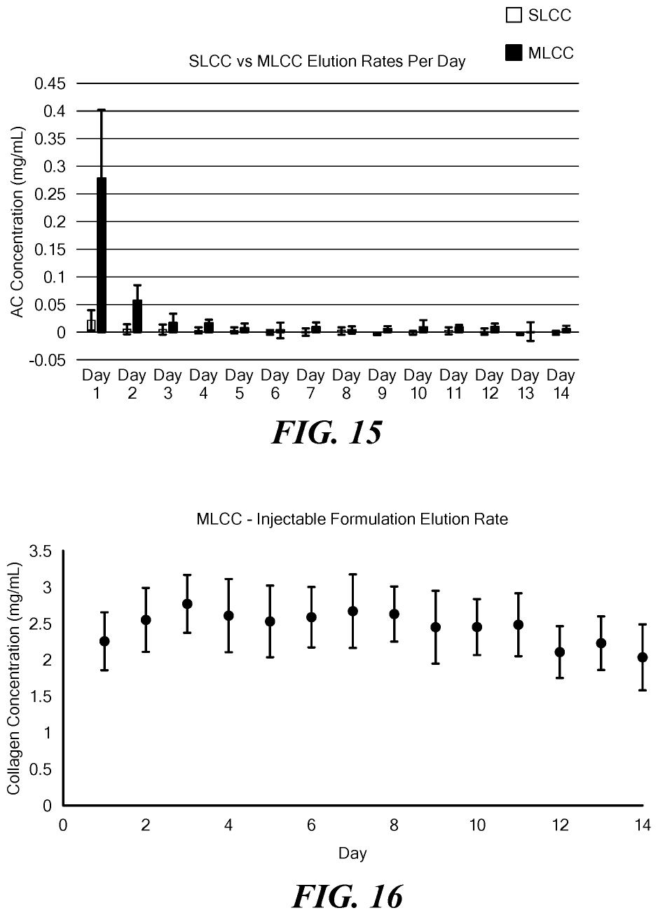

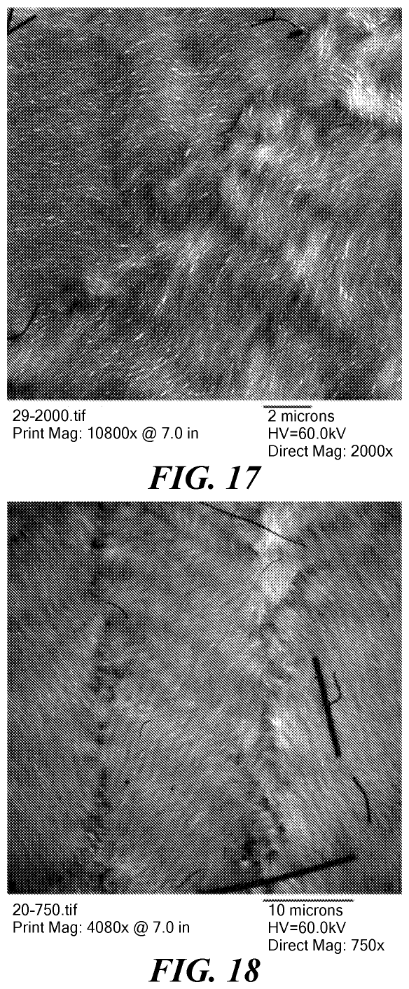

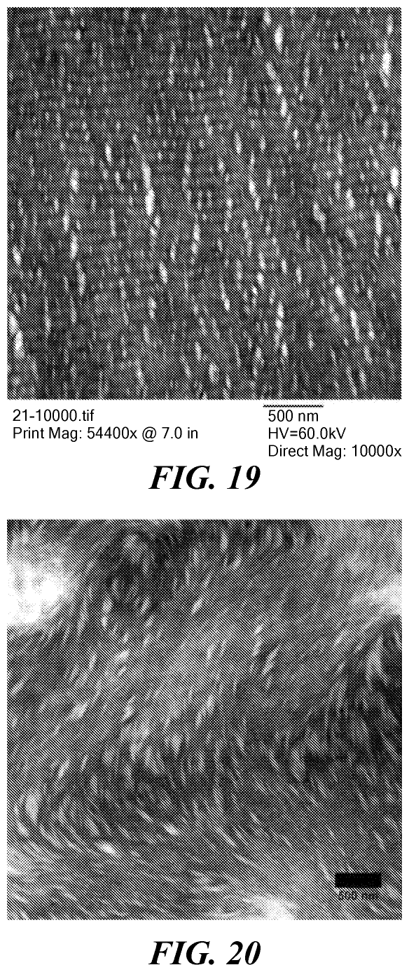

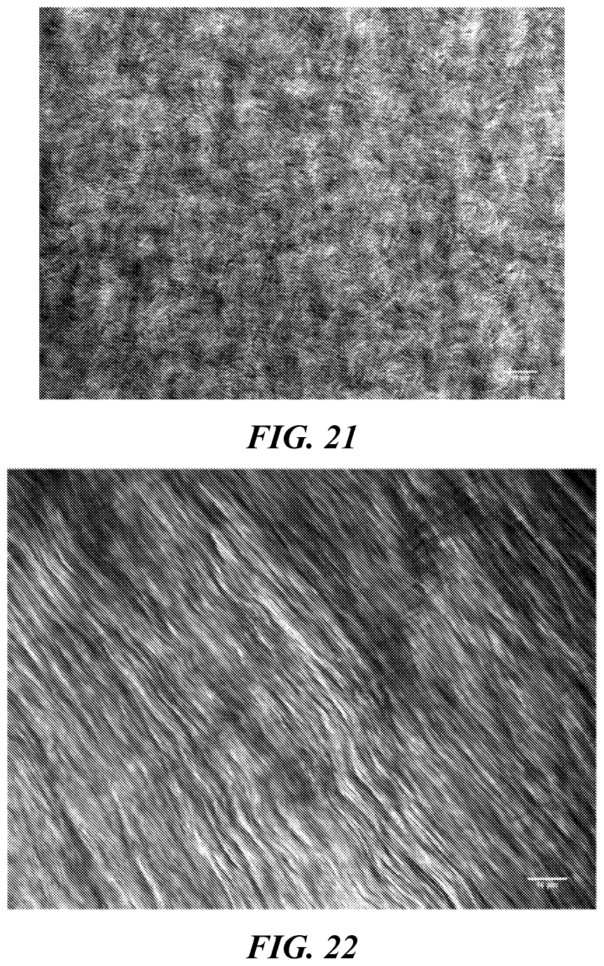

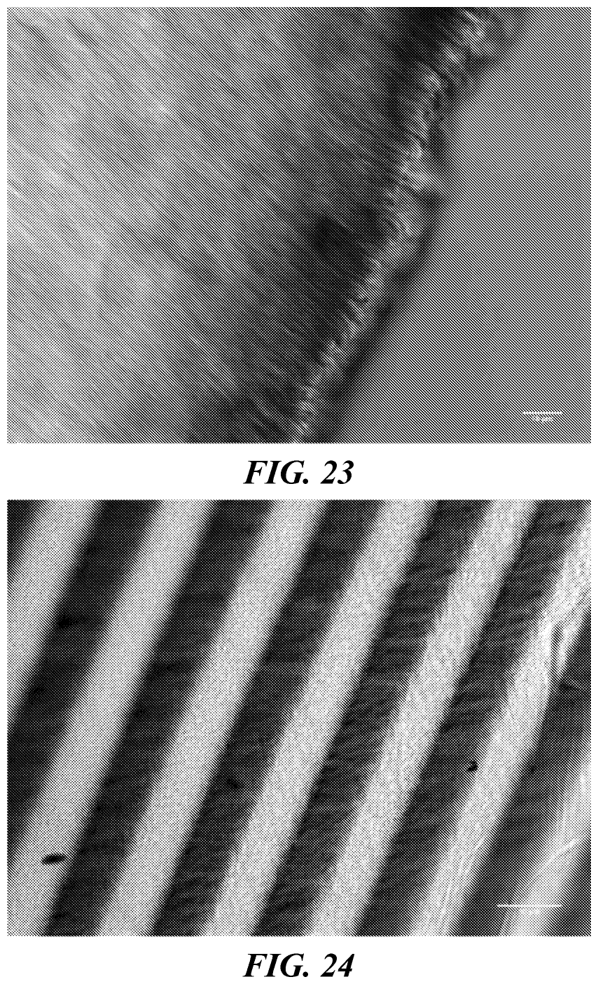

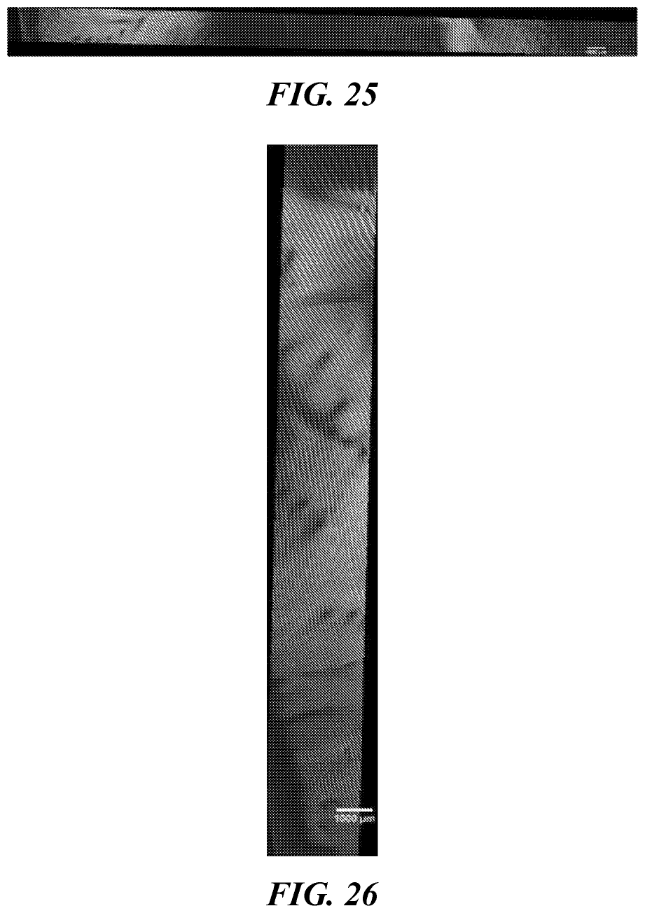

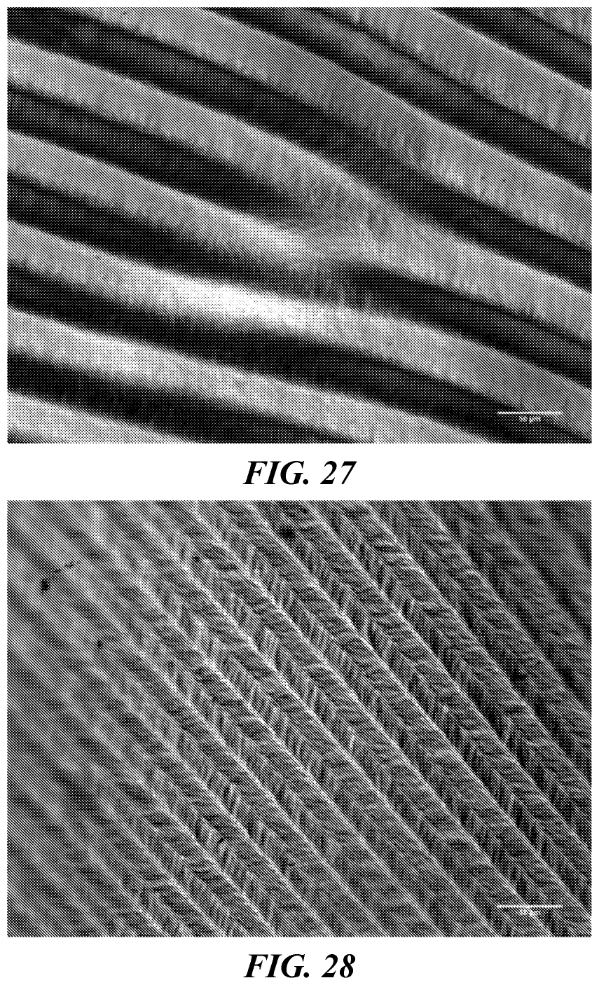

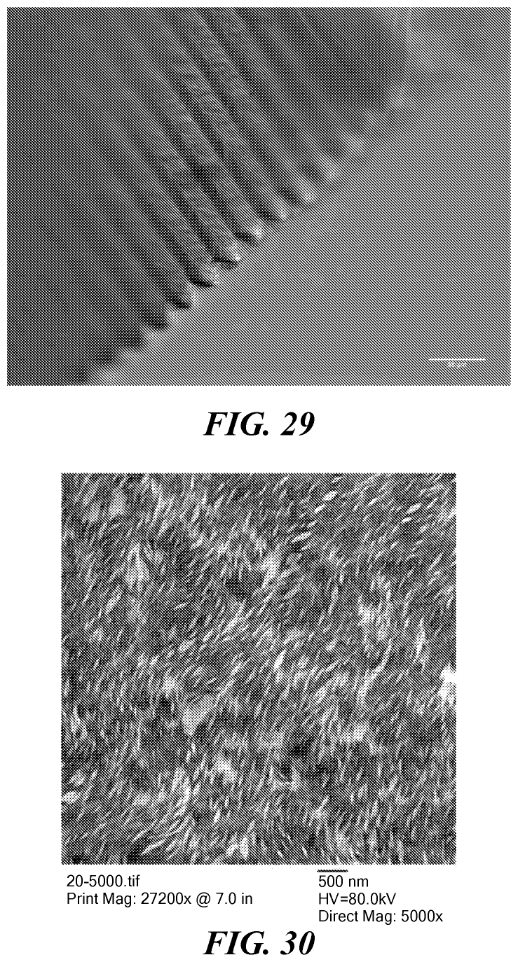

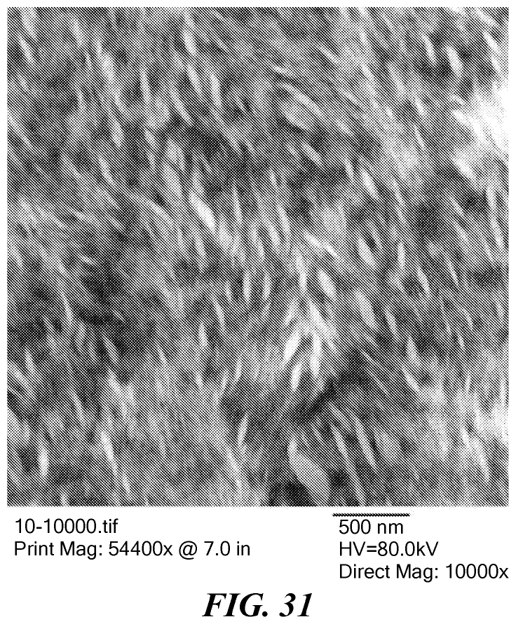

Partial transection model: To simulate a focal defect, tissue models are partially transected to produce a central linear defect of 50% of the width of the strip (FIGS. 9A-9D).

Repair procedure: Bi-stable patches containing dense AC fabricated with labeled monomers (tropocollagen) are placed on the DDCS strip (or wrapped around the RTT), with the MLCC in direct contact with the “damaged tissue”. Specimens are mounted in a mechanobioreactor. Tensile strain is applied as a linear ramp at strain rates of 0.1, 1 and 10/s to 8% engineering strain, under the linear elastic limit of DDCS and RTT. Zero-strain and 8% static strain serve as controls for loading and for dynamic loading. The strain rate values are based on muscle contraction velocities and extensional strain rates required to induce collagen assembly. Strain is held at 8% for 5 minutes to permit ample time for re-equilibration of the collagen monomers with the sample and for viscous relaxation.

Quantitative determination of AC transfer: Tendon models are assessed via fluorescence imaging to quantify amount of fluorescently labeled collagen transferred to the repair site. Results are expressed as area labeled collagen (normalized for labelled collagen ratio)/area total collagen. In both partial transection models, the extensional strain rate varies from the center of the defect to the edges of the sample and along the sample's long axis, allowing comparison of the effect of varying extensional strain rates on collagen incorporation.

Mechanical testing: Following fluorescence imaging, samples are subjected to the mechanical testing protocol described above. Mechanical properties of repair outcomes are compared against mechanical properties of damaged tissue models.

### Example 5. Testing of Immune Responses to MLCCS and SLCCS in an Animal Model

The in vivo safety of MLCCS, SLCCS, and the bi-stable MLCC/SLCC rotator cuff patch is tested in a rabbit model. Immune and inflammatory responses in the tendon and local tissues are assessed, as well as any gross tissue changes.

Optimization of the patch configuration, location and fixation. Twelve shoulders from six New Zealand White (NZW) rabbit carcasses are used to test the patch position, fixation and retention on the supraspinatus tendon by articulating the joint mechanically for 12 hours while immersed in PBS in a simplified custom version of the Admet Mechanobioreactor.

NZW rabbits undergo bilateral shoulder surgery to assess the local cellular, neovascular and immune response of three collagen releasing patches at days 7, 14 and 28 on the subscapularis tendon and the surrounding tissues. Sham surgeries on contralateral shoulders serve as control. At the time of harvest the supraspinatus tendon and surrounding tissues are photographed to document the gross tissue response. Retrieved tendons are harvested, fixed in 10% NBF at 4° C., dehydrated and processed by standard paraffin embedding. Blocks with tendons are prepared for longitudinal and cross section sectioning to study the tendon, patch and their interface. Three 5 μm sections are obtained from each block and for longitudinal sections, three 5 μm sections are obtained at 2 tendon depths (sub-surface and mid-depth) and stained with H&E, Masson's trichrome and Picrosirius red. Adjacent tissues are also harvested and analyzed.

### Example 6. Acceleration of Tendon Repair by SLCC/MLCC Patch in an Animal Model

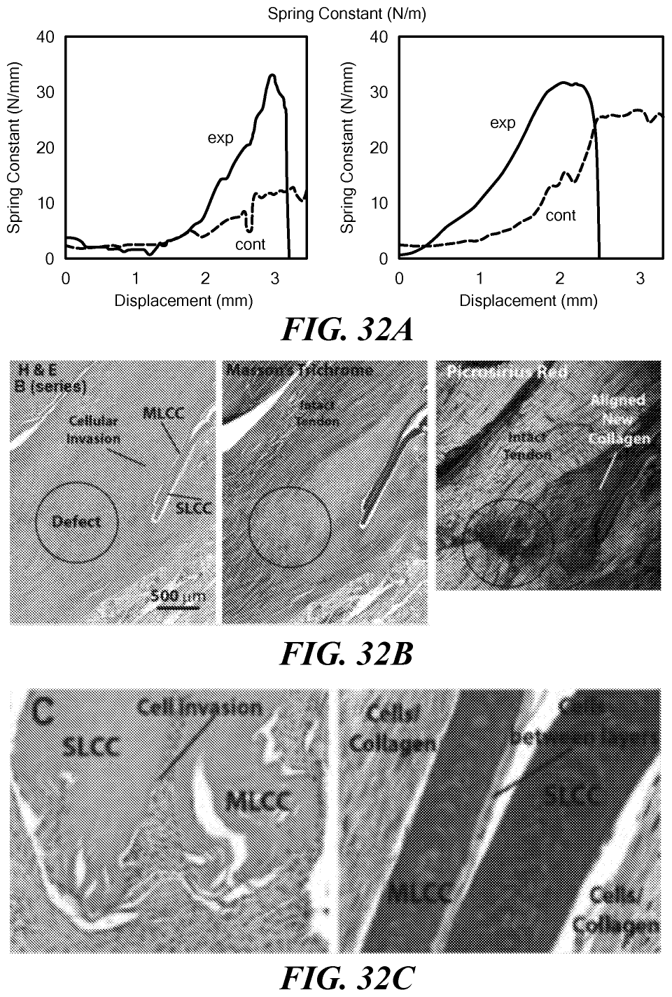

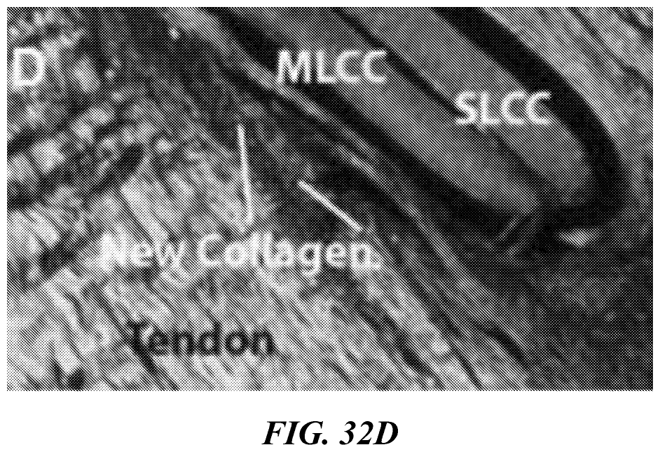

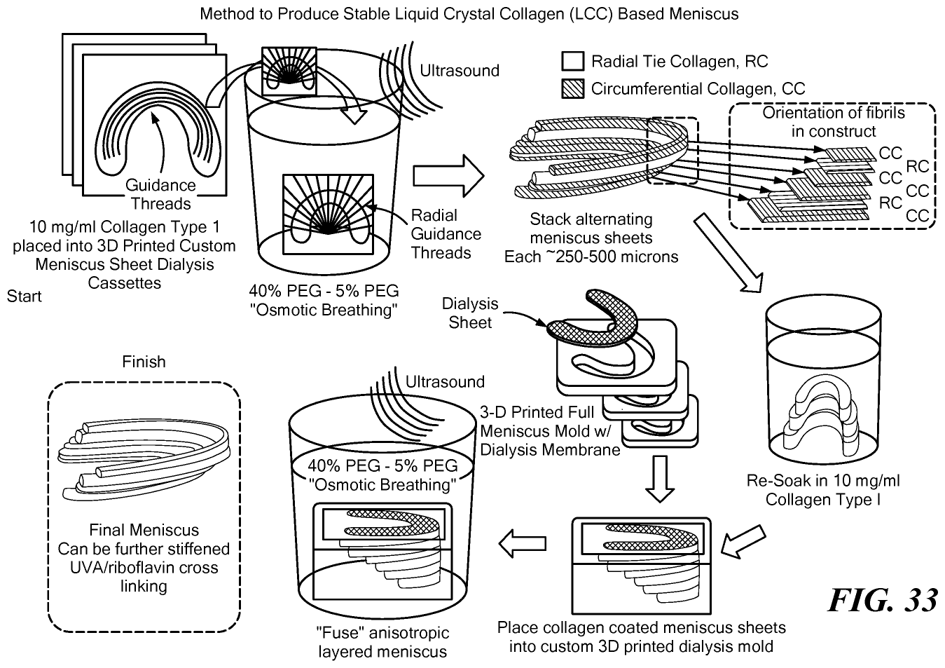

The punch model of Achilles tendon repair in rats was used to test the effectiveness of SLCC/MLCC bistable collagen patches in promoting tendon repair. A cylindrical defect was created in rat Achilles tendon using a punch, and treatment was induced in a group of the animals by placing a bistable SLCC/MLCC collagen implant within about 0.5-2 mm of the defect. The implant was aligned with the direction of collagen fibers in the tendon. After 14 days, tendons were removed from a treated group and an untreated group and their mechanical properties analyzed. The results shown in FIG. 32A show a dramatic increase in stiffness, as measured by the spring constant, in the treated animals (exp) compared to untreated controls (cont). Histology images showed aligned new collagen formed in the space between the implant and the defect as well as disassociation of the SLCC and MLCC materials of the implant (FIG. 32B). Higher magnification images (FIGS. 32C, 32D) revealed infiltration of cells into and surrounding the implant and association of cells with new collagen near the implant. This experiment confirmed that implanting a patch containing aligned SLCC and MLCC material can accelerate repair of damaged tendon and in particular lead to a much shorter time to loadbearing condition.

### Example 7. Implantable/Injectable Collagen In Vivo Ultrasound Conversion of MLCC to SLCC for Stability

MLCC and SLCC are implanted or injected (after being fragmented) to serve as a tissue filler in a subject. Following injection, ultrasound is applied to the subject to convert the MLCC to SLCC and fuse the particles into a more solid, degradation-resistant state that possesses an overall lower energy.

### REFERENCES

1. Colvin, A. C., Egorova, N., Harrison, A. K., Moskowitz, A. & Flatow, E. L. National trends in rotator cuff repair. J Bone Joint Surg Am 94, 227-233 (2012).
2. Crawford, M. Does Age Affect Surgically Repaired Rotator Cuffs? AAOS Now 8, 3 (2014).
3. Csintalan, R. P., Mack), M. C. & Funahashi, T. T. Incidence rate of anterior cruciate ligament reconstructions. Perm J 12, 17-21 (2008).
4. de Jong, J. P. et al. The incidence of acute traumatic tendon injuries in the hand and wrist: a 10-year population-based study. Clin Orthop Surg 6, 196-202 (2014).
5. Ensor, K. L., Kwon, Y. W., Dibeneditto, M. R., Zuckerman, J. D. & Rokito, A. S. The rising incidence of rotator cuff repairs. J Shoulder Elbow Surg 22, 1628-1632 (2013).
6. Glazebrook, M. New guideline addresses acute Achilles tendon ruptures. AAOS Now 4, 4 (2010).
7. Huttunen, T. T., Kannus, P., Rolf, C., Fellander-Tsai, L. & Mattila, V. M. Acute Achilles Tendon Ruptures: Incidence of Injury and Surgery in Sweden Between 2001 and 2012. The American journal of sports medicine (2014).
8. Lantto, I., Heikkinen, J., Flinkkila, T., Ohtonen, P. & Leppilahti, J. Epidemiology of Achilles tendon ruptures: Increasing incidence over a 33-year period. Scand J Med Sci Sports (2014).
9. Mall, N. A. et al. Incidence and Trends of Anterior Cruciate Ligament Reconstruction in the United States. The American journal of sports medicine (2014).
10. Papalia, R. et al. Augmentation techniques for rotator cuff repair. Br Med Bull 105, 107-138 (2013).
11. Rawson, S., Cartmell, S. & Wong, J. Suture techniques for tendon repair; a comparative review. Muscles Ligaments Tendons J 3, 220-228 (2013).
12. Cohen, S. P. et al. Diagnoses and factors associated with medical evacuation and return to duty for service members participating in Operation Iraqi Freedom or Operation Enduring Freedom: a prospective cohort study. Lancet 375, 301-309, doi:10.1016/50140-6736(09)61797-9 (2010).
13. Konrath, G. A. et al. Outcomes following repair of quadriceps tendon ruptures. Journal of orthopaedic trauma 12, 273-279 (1998).
14. Almekinders, L. C. & Almekinders, S. V. Outcome in the treatment of chronic overuse sports injuries: a retrospective study. The Journal of orthopaedic and sports physical therapy 19, 157-161, doi:10.2519/jospt.1994.19.3.157 (1994).
15. Clayton, R. A. & Court-Brown, C. M. The epidemiology of musculoskeletal tendinous and ligamentous injuries. Injury 39, 1338-1344, doi:10.1016/j.injury.2008.06.021 (2008).
16. Charousset, C., Bellaiche, L., Kalra, K. & Petrover, D. Arthroscopic repair of full-thickness rotator cuff tears: is there tendon healing in patients aged 65 years or older? Arthroscopy: the journal of arthroscopic & related surgery: official publication of the Arthroscopy Association of North America and the International Arthroscopy Association 26, 302-309, doi:10.1016/j.arthro.2009.08.027 (2010).
17. Yang, G., Rothrauff, B. B. & Tuan, R. S. Tendon and ligament regeneration and repair: clinical relevance and developmental paradigm. Birth defects research. Part C, Embryo today: reviews 99, 203-222, doi:10.1002/bdrc.21041 (2013).
18. Gulati, V. et al. Management of achilles tendon injury: A current concepts systematic review. World J Orthop 6, 380-386, doi:10.5312/wjo.v6.i4.380 (2015).
19. Khan, R. J. & Carey Smith, R. L. Surgical interventions for treating acute Achilles tendon ruptures. Cochrane Database Syst Rev, CD003674, doi:10.1002/14651858.CD003674.pub4 (2010).
20. McElvany, M. D., McGoldrick, E., Gee, A. O., Neradilek, M. B. & Matsen, F. A., 3rd. Rotator cuff repair: published evidence on factors associated with repair integrity and clinical outcome. The American journal of sports medicine 43, 491-500, doi:10.1177/0363546514529644 (2015).
21. LaPrade, R. F. et al. Biologic Treatments for Sports Injuries II Think Tank-Current Concepts, Future Research, and Barriers to Advancement, Part 1: Biologics Overview, Ligament Injury, Tendinopathy. The American journal of sports medicine, doi:10.1177/0363546516634674 (2016).
22. Snedeker, J. G. & Foolen, J. Tendon injury and repair—A perspective on the basic mechanisms of tendon disease and future clinical therapy. Acta biomaterialia 63, 18-36, doi:10.1016/j.actbio.2017.08.032 (2017).
23. Bhole, A. P. et al. Mechanical strain enhances survivability of collagen micronetworks in the presence of collagenase: implications for load-bearing matrix growth and stability. Philosophical transactions. Series A, Mathematical, physical, and engineering sciences 367, 3339-3362, doi:10.1098/rsta.2009.0093 (2009).
24. Paten, J. A. et al. Flow-Induced Crystallization of Collagen: A Potentially Critical Mechanism in Early Tissue Formation. ACS nano 10, 5027-5040, doi:10.1021/acsnano.5b07756 (2016).
25. Musculoskeletal Disorders and the Workplace: Low Back and Upper Extremities. (The National Academies Press, 2001).
26. AAOS. Burden of Musculoskeletal Diseases in the United States: Prevalence, Societal and Economic Cost. Second edn, (American Academy of Orthopaedic Surgeons, 2011).
27. Muller, S. A., Todorov, A., Heisterbach, P. E., Martin, I. & Majewski, M. Tendon healing: an overview of physiology, biology, and pathology of tendon healing and systematic review of state of the art in tendon bioengineering. Knee Surg Sports Traumatol Arthrosc (2013).
28. Paterno, M. V., Ford, K. R., Myer, G. D., Heyl, R. & Hewett, T. E. Limb asymmetries in landing and jumping 2 years following anterior cruciate ligament reconstruction. Clin J Sport Med 17, 258-262 (2007).
29. Schepull, T., Kvist, J., Andersson, C. & Aspenberg, P. Mechanical properties during healing of Achilles tendon ruptures to predict final outcome: a pilot Roentgen stereophotogrammetric analysis in 10 patients. BMC Musculoskelet Disord 8, 116 (2007).
30. Tibone, J. E. & Antich, T. J. A biomechanical analysis of anterior cruciate ligament reconstruction with the patellar tendon. A two year followup. The American journal of sports medicine 16, 332-335 (1988).
31. Galatz, L. M., Ball, C. M., Teefey, S. A., Middleton, W. D. & Yamaguchi, K. The outcome and repair integrity of completely arthroscopically repaired large and massive rotator cuff tears. J Bone Joint Surg Am 86-A, 219-224 (2004).
32. Koh, K. H., Kang, K. C., Lim, T. K., Shon, M. S. & Yoo, J. C. Prospective randomized clinical trial of single-versus double-row suture anchor repair in 2- to 4-cm rotator cuff tears: clinical and magnetic resonance imaging results. Arthroscopy: the journal of arthroscopic & related surgery: official publication of the Arthroscopy Association of North America and the International Arthroscopy Association 27, 453-462, doi:10.1016/j.arthro.2010.11.059 (2011).
33. Parnes, N., DeFranco, M., Wells, J. H., Higgins, L. D. & Warner, J. J. Complications after arthroscopic revision rotator cuff repair. Arthroscopy: the journal of arthroscopic & related surgery: official publication of the Arthroscopy Association of North America and the International Arthroscopy Association 29, 1479-1486, doi:10.1016/j.arthro.2013.06.015 (2013).
34. Saridakis, P. & Jones, G. Outcomes of single-row and double-row arthroscopic rotator cuff repair: a systematic review. J Bone Joint Surg Am 92, 732-742, doi:10.2106/JBJS.I.01295 (2010).
35. Tashjian, R. Z. et al. Factors affecting healing rates after arthroscopic double-row rotator cuff repair. The American journal of sports medicine 38, 2435-2442, doi:10.1177/0363546510382835 (2010).
36. Toussaint, B., Schnaser, E., Bosley, J., Lefebvre, Y. & Gobezie, R. Early structural and functional outcomes for arthroscopic double-row transosseous-equivalent rotator cuff repair. The American journal of sports medicine 39, 1217-1225, doi:10.1177/0363546510397725 (2011).
37. Cheung, E. V., Silverio, L. & Sperling, J. W. Strategies in biologic augmentation of rotator cuff repair: a review. Clin Orthop Relat Res 468, 1476-1484 (2010).
38. Kannus, P. & Jozsa, L. Histopathological changes preceding spontaneous rupture of a tendon. A controlled study of 891 patients. J Bone Joint Surg Am 73, 1507-1525 (1991).
39. F G, L. Shrinkage of Collagen. Biochimica et Biophysica Acta 3, 170-187 (1949).
40. Huang, C. & Yannas, I. V. Mechanochemical studies of enzymatic degradation of insoluble collagen fibers. Journal of biomedical materials research 11, 137-154, doi:10.1002/jbm.820110113 (1977).
41. Nabeshima, Y., Grood, E. S., Sakurai, A. & Herman, J. H. Uniaxial tension inhibits tendon collagen degradation by collagenase in vitro. Journal of orthopaedic research: official publication of the Orthopaedic Research Society 14, 123-130, doi:10.1002/jor.1100140120 (1996).
42. Camp, R. J. et al. Molecular mechanochemistry: low force switch slows enzymatic cleavage of human type I collagen monomer. Journal of the American Chemical Society 133, 4073-4078 (2011).
43. Flynn, B. P. et al. Mechanical strain stabilizes reconstituted collagen fibrils against enzymatic degradation by mammalian collagenase matrix metalloproteinase 8 (MMP-8). PloS one 5, e12337, doi:10.1371/joumal.pone.0012337 (2010).
44. Flynn, B. P., Tilburey, G. E. & Ruberti, J. W. Highly sensitive single-fibril erosion assay demonstrates mechanochemical switch in native collagen fibrils. Biomechanics and modeling in mechanobiology 12, 291-300, doi:10.1007/s10237-012-0399-2 (2013).
45. Wyatt, K. E., Bourne, J. W. & Torzilli, P. A. Deformation-dependent enzyme mechanokinetic cleavage of type I collagen. Journal of biomechanical engineering 131, 051004, doi:10.1115/1.3078177 (2009).
46. Zareian, R. et al. Probing collagen/enzyme mechanochemistry in native tissue with dynamic, enzyme-induced creep. Langmuir: the ACS journal of surfaces and colloids 26, 9917-9926 (2010).
47. Ruberti, J. W. & Hallab, N. J. Strain-controlled enzymatic cleavage of collagen in loaded matrix. Biochemical and biophysical research communications 336, 483-489, doi:10.1016/j.bbrc.2005.08.128 (2005).
48. Saeidi, N., Sander, E. A. & Ruberti, J. W. Dynamic shear-influenced collagen self-assembly. Biomaterials 30, 65816592, doi:10.1016/j.biomaterials.2009.07.070 (2009).
49. Saeidi, N., Sander, E. A., Zareian, R. & Ruberti, J. W. Production of highly aligned collagen lamellae by combining shear force and thin film confinement. Ada biomaterialia 7, 2437-2447, doi:10.1016/j.actbio.2011.02.038 (2011).
50. Saeidi, N. et al. Disorganized collagen scaffold interferes with fibroblast mediated deposition of organized extracellular matrix in vitro. Biotechnology and bioengineering 109, 2683-2698, doi:10.1002/bit.24533 (2012).
51. Saeidi, N. et al. Molecular crowding of collagen: a pathway to produce highly-organized collagenous structures. Biomaterials 33, 7366-7374, doi:10.1016/j.biomaterials.2012.06.041 (2012).
52. Paten, J. A. et al. Utility of an optically-based, micromechanical system for printing collagen fibers. Biomaterials 34, 2577-2587, doi:10.1016/j.biomaterials.2012.12.028 (2013).
53. Zhou, E. H. et al. Assessing the impact of engineered nanoparticles on wound healing using a novel in vitro bioassay. Nanomedicine (Lond) 9, 2803-2815, doi:10.2217/nnm.14.40 (2014).
54. Susilo, M. E., Paten, J. A., Sander, E. A., Nguyen, T. D. & Ruberti, J. W. Collagen network strengthening following cyclic tensile loading. Interface focus 6, 20150088, doi:10.1098/rsfs.2015.0088 (2016).
55. Ejim, O. S., Blunn, G. W. & Brown, R. A. Production of artificial-orientated mats and strands from plasma fibronectin: a morphological study. Biomaterials 14, 743-748 (1993).
56. Brown, R. A., Blunn, G. W. & Ejim, 0. S. Preparation of orientated fibrous mats from fibronectin: composition and stability. Biomaterials 15, 457-464 (1994).
57. Wang, Z., Ma, Z. & Li, L. Flow-Induced Crystallization of Polymers: Molecular and Thermodynamic Considerations. Macromolecules 49, 13 (2016).
58. Stopak, D., Wessells, N. K. & Harris, A. K. Morphogenetic rearrangement of injected collagen in developing chicken limb buds. Proc Natl Acad Sci USA 82, 2804-2808 (1985).
59. Suh, D. S. et al. Atelocollagen Enhances the Healing of Rotator Cuff Tendon in Rabbit Model. The American journal of sports medicine 45, 2019-2027, doi:10.1177/0363546517703336 (2017).
60. Martinez-Ortiz M A, H.-F. A., Pimentel-Gonzalez D J, Compos-Montiel R G, Vargas-Torres A, Aguirre-Alvarez G. Extraction and characterization of collagen from rabbit skin: partial characterization. Journal of Food 13, 253-258 (2015).
61. Wollensak, G., Spoerl, E. & Seiler, T. Stress-strain measurements of human and porcine corneas after riboflavin-ultraviolet-A-induced cross-linking. Journal of cataract and refractive surgery 29, 1780-1785 (2003).
62. Delgado, L. M., Bayon, Y., Pandit, A. & Zeugolis, D. I. To cross-link or not to cross-link? Cross-linking associated foreign body response of collagen-based devices. Tissue engineering. Part B, Reviews 21, 298-313, doi:10.1089/ten.TEB.2014.0290 (2015).
63. Maganaris, C. N. & Paul, J. P. In vivo human tendon mechanical properties. The Journal of physiology 521 Pt 1, 307-313 (1999).
64. Maganaris, C. N. & Paul, J. P. Tensile properties of the in vivo human gastrocnemius tendon. J Biomech 35, 16391646 (2002).
65. Wren, T. A., Yerby, S. A., Beaupre, G. S. & Carter, D. R. Mechanical properties of the human achilles tendon. Clinical biomechanics 16, 245-251 (2001).
66. Makareeva, E., Cabral, W. A., Marini, J. C. & Leikin, S. Molecular mechanism of alpha 1(l)-osteogenesis imperfecta/Ehlers-Danlos syndrome: unfolding of an N-anchor domain at the N-terminal end of the type I collagen triple helix. The Journal of biological chemistry 281, 6463-6470, doi:10.1074/jbc.M511830200 (2006).
67. Reddy, P. S., Idamakanti, N., Derbyshire, J. B. & Nagy, E. Porcine adenoviruses types 1, 2 and 3 have short and simple early E-3 regions. Virus research 43, 99-109 (1996).
68. Durgam, S. S., Stewart, A. A., Sivaguru, M., Wagoner Johnson, A. J. & Stewart, M. C. Tendon-derived progenitor cells improve healing of collagenase-induced flexor tendinitis. Journal of orthopaedic research: official publication of the Orthopaedic Research Society 34, 2162-2171, doi:10.1002/jor.23251 (2016).
69. Hsu, R. W., Hsu, W. H., Tai, C. L. & Lee, K. F. Effect of hyperbaric oxygen therapy on patellar tendinopathy in a rabbit model. The Journal of trauma 57, 1060-1064 (2004).
70. Kamineni, S., Butterfield, T. & Sinai, A. Percutaneous ultrasonic debridement of tendinopathy—a pilot Achilles rabbit model. Journal of orthopaedic surgery and research 10, 70, doi:10.1186/s13018-015-0207-7 (2015).
71. Puxkandl, R. et al. Viscoelastic properties of collagen: synchrotron radiation investigations and structural model. Philosophical transactions of the Royal Society of London. Series B, Biological sciences 357, 191-197, doi:10.1098/rstb.2001.1033 (2002).
72. Rigby B J, H. N., Spikes J D, Eyring H. The mechanical properties of rat tail tendon. Journal of

General Physiology 43, 265-283 (1959).
73. Olson, C. B. & Swett, C. P., Jr. Speed of contraction of skeletal muscle. The effect of hypoactivity and hyperactivity. Archives of neurology 20, 263-270 (1969).
74. Martins, C. A. et al. Dextrose prolotherapy and corticosteroid injection into rat Achilles tendon. Knee Surg Sports Traumatol Arthrosc 20, 1895-1900, doi:10.1007/s00167-011-1789-1 (2012).
75. Flint, M. H., Lyons, M. F., Meaney, M. F. & Williams, D. E. The Masson staining of collagen—an explanation of an apparent paradox. The Histochemical Journal 7, 529-546, doi:10.1007/bf01003791 (1975).
76. Martinello, T. et al. Might the Masson trichrome stain be considered a useful method for categorizing experimental tendon lesions? Histol Histopathol 30, 963-969, doi:10.14670/HH-11-601 (2015).

1. A non-naturally occurring, stable, liquid crystal collagen composition, wherein the composition comprises type I, II, III, V, and/or XI collagen organized into fibril-like structures 50-200 nm in diameter, wherein the fibril-like structures are locally aligned along their longitudinal axis and have D-periodic banding structure larger than about 67 nm, and wherein the composition has a density of at least about 500 mg/mL.

2. The collagen composition of claim 1, wherein the fibril-like structures shed monomeric collagen at a rate less than 1% per day at 37° C.

3. The collagen composition of claim 1, wherein the composition has a density of about 800 mg/mL.

4. A non-naturally occurring, metastable, liquid crystal collagen composition, wherein the composition comprises type I, II, III, V, and/or XI collagen organized into fibril-like structures 50-100 nm in diameter, wherein the fibrils are locally aligned along their longitudinal axis and substantially lack native D-periodic banding structure, and wherein the composition has a density in the range from about 100 to about 500 mg/mL.

5. A bi-stable liquid crystal collagen composition comprising the composition of claim 1 and the composition of claim 4.

6. The bi-stable liquid crystal collagen composition of claim 5, wherein the relative amounts of the composition of claim 1 and the composition of claim 4 are selected to provide a desired collagen delivery rate in a collagen assembly process.

7. The composition of any one of claims 1-6 configured as a plurality of particles, a gel, or a tissue scaffold.

8. The composition of claim 7 configured as a tissue scaffold, wherein the scaffold further comprises one or more types of cells and/or one or more therapeutic agents.

9. The composition of claim 8, wherein the one or more therapeutic agents are selected from the group consisting of anti-inflammatory molecules, matrix anabolic molecules (signaling molecules which drive matrix assembly e.g. TGF-β3 and molecules directly involved in matrix construction e.g. fibronectin), matrix catabolic molecules (signaling molecules which drive matrix destruction e.g. IL-1 and molecules directly involved in matrix destruction e.g. matrix metalloproteases), proteoglycans, and glycosaminoglycans.

10. A method of making a stable liquid crystalline collagen composition, comprising the steps of:
(a) concentrating a solution comprising type I, II, III, V, and/or XI collagen under acidic conditions;
(b) subjecting the product of (a) to periodic sonication for several days;
(c) osmotically cycling, neutralizing, and warming the product of (b) to form the stable liquid crystalline collagen composition.

11. The method of claim 10, wherein step (a) comprises dialyzing the collagen solution against an aqueous solution comprising about 10% polyethylene glycol having a molecular weight of about 35000 Daltons at a pH of about 1.5-2.5.

12. The method of claim 11, wherein step (a) comprises concentrating the collagen solution to about 10 to about 15 mg/mL.

13. The method of claim 11 or 12, further comprising the step of:
(a1) dialyzing the product of step (a) against an aqueous solution comprising about 40% polyethylene glycol having a molecular weight of about 35000 Daltons at a pH of about 1.5-2.5 for about 7 days.

14. The method of any of claims 10-13, wherein step (a) is performed at about 4° C.

15. The method of claim 10, wherein step (b) comprises subjecting the concentrating collagen solution to sonication for at least 10 minutes at least twice per day.

16. The method of claim 10, wherein the osmotic cycling of step (c) comprises alternating dialysis of the concentrating collagen solution against (i) 40% polyethylene glycol in phosphate buffered saline at pH 7.4 and (ii) phosphate buffered saline at pH 7.4.

17. The method of any of claims 10-16, wherein warming of step (c) comprises performing said osmotic cycling and neutralization at about 35° C.

18. The method of any of claims 10-17, wherein the stable liquid crystalline collagen is stable under conditions of mechanical load, enzymatic cleavage, and solvent dissolution.

19. A method of making a metastable liquid crystalline collagen composition, comprising the steps of:
(a) concentrating a solution comprising type I, II, III, V, and/or XI collagen under acidic conditions;
(b) neutralizing and warming the product of (a) to form the metastable liquid crystalline collagen composition.

20. The method of claim 19, wherein step (a) comprises dialyzing the collagen solution against an aqueous solution comprising about 10% polyethylene glycol having a molecular weight of about 35000 Daltons at a pH of about 1.5-2.5.

21. The method of claim 20, wherein step (a) comprises concentrating the collagen solution to about 10 to about 15 mg/mL.

22. The method of claim 20 or 21, further comprising the step of:
(a1) dialyzing the product of step (a) against an aqueous solution comprising about 40% polyethylene glycol having a molecular weight of about 35000 Daltons at a pH of about 1.5-2.5 for about 7 days.

23. The method of any of claims 19-22, wherein step (a) is performed at about 4° C.

24. The method of any of claims 19-23, wherein step (b) comprises dialysis of the concentrating collagen solution against 40% polyethylene glycol in phosphate buffered saline at pH 7.4.

25. The method of any of claims 19-24, wherein the warming of step (b) comprises performing said neutralization at about 35° C.

26. The method of any of claims 10-25, further comprising adding one or more anti-inflammatory molecules, matrix anabolic molecules, matrix catabolic molecules, proteoglycans, and/or glycosaminoglycans to the collagen solution at any step of the method.

27. The method of any of claims 10-26, further comprising applying tensile loading to the composition at any step of the method, optionally with the addition of monomeric type I, II, III, V, and/or XI collagen to the composition.

28. A method to aid in repairing damaged connective tissue in a human or other mammalian subject, the method comprising the steps of:
(a) providing a liquid crystalline collagen composition of any of claims 1-9; and
(b) placing said composition into a region comprising damaged connective tissue in the subject;
whereby said damaged connective tissue is at least partially repaired.

29. The method of claim 28, wherein step (a) comprises:
(a1) obtaining cells from the subject;
(a2) culturing the cells and optionally inducing differentiation and/or proliferation of the cells and/or collagen secretion by the cells;
(a3) harvesting collagen from the culture; and
(a4) forming a liquid crystalline collagen composition of any of claims 1-9 using the harvested collagen.

30. The method of claim 28 or 29, further comprising the step of:
(c) performing mechanical stimulatory therapy of said region.

31. The method of claim 30, wherein said mechanical stimulatory therapy comprises one or more of continuous passive motion, ultrasound, vibration, and electromyostimulation.

32. The method of any of claims 28-31, wherein step (b) is performed continuously or intermittently over a period of time.

33. The method of any of claims 28-32, wherein the liquid crystalline collagen composition further comprises one or more additional extracellular matrix molecular component.

34. The method of claim 33, wherein the additional extracellular matrix molecular component is a protein selected from the group consisting of other forms of fibrillar collagen, elastin, and fibronectin.

35. The method of claim 34, wherein the type I, II, III, V collagen, other form of fibrillar collagen, elastin, and/or fibronectin are obtained from a different species than the human or other mammalian subject.

36. The method of claim 34, wherein the type I, II, III, V collagen, other form of fibrillar collagen, elastin, and/or fibronectin are obtained from stem cells isolated from the subject.

37. The method of any of claims 28-36, wherein the damaged connective tissue comprises a wound, broken or fractured bone, ruptured tendon, hernia, damaged barrier membrane, or inflammation of a connective tissue.

38. The method of any of claims 28-37, wherein the method speeds repair of the damaged connective tissue.

39. A method of altering a chemical or physical property of the liquid crystalline collagen composition of any one of claims 1-9, the method comprising: (i) adding the composition to a scaffold; (ii) stacking rolling, or bending the composition; or (iii) mineralizing the composition with calcium phosphate.

40. A method of growing cells in culture, the method comprising adding the liquid crystalline collagen composition of any one of claims 1-9 to a cell culture, whereby cells in the culture become bound to or aligned with fibril-like structures in the composition.

41. A medical device comprising the liquid crystalline collagen composition of any one of claims 1-9

42. A tissue scaffold comprising the liquid crystalline collagen composition of any one of claims 1-9.

43. The tissue scaffold of claim 42, further comprising a plurality of cells, preferably stem cells, and more preferably mesenchymal stem cells.

44. The composition of claim 4, wherein the fibril-like structures further comprise contrasting layered bands having a layer tilt angle of ˜45° compared to a longitudinal axis of the fibril-like structures, the bands comprising chevron-like defect structures and a chevron-like interface in a center of the structures.
